(19) United States
(12) Patent Application Publication
(10) Pub. No.: US 2004/0106168 A1
(43) Pub. Date: Jun. 3, 2004

(54) System and method for neuronal network analysis

(76) Inventor: Daron G. Evans, Dallas, TX (US)

Correspondence Address: CHALKER FLORES, LLP, 12700 PARK CENTRAL, STE. 455, DALLAS, TX, 75251, US

(21) Appl. No.: 10/370,786

(22) Filed: Feb. 20, 2003

Related U.S. Application Data
(60) Provisional application No. 60/430,409, filed on Dec. 2, 2002.

(51) Int. Cl. G01N 33/48; C12M 1/00

(52) U.S. Cl. 435/40.5; 435/29; 435/283.1

(57) ABSTRACT

The present invention provides a system and method for testing the neuronal effects of a compound. The system (100) includes a microelectrode array (102), a data capture unit (106) communicably coupled to the microelectrode array (102), a processor (108) communicably coupled to the data capture unit (106) and one or more input/output devices (110) communicably coupled to the processor (108). The microelectrode array (102) is capable of supporting genetically modified neuronal cells (104) and measuring neuronal activity. The method (400) determines the effects of a sample on genetically modified neuronal cells by growing a culture of genetically modified neuronal cells on a microelectrode array (402) and exposing a portion of the genetically modified neuronal cells to a sample (404). The effects of the sample on the genetically modified neuronal cells exposed to the sample are measured to determine the effects of the sample on the genetically modified neuronal cells (406).

### TECHNICAL FIELD OF THE INVENTION

The present invention relates in general to the field of action potential analysis, and more particularly, to the use of advanced neuronal networks detection techniques for the detailed analysis of neuronal signal transduction pathways and their use for large-scale reproducible analysis.

### BACKGROUND OF THE INVENTION

This application claims priority to U.S. Provisional Patent Application Serial No. 60/430,409, filed Dec. 2, 2002. Without limiting the scope of the invention, the background of the invention is described in connection with the recording and analysis of neuronal action potentials using substrate integrated, thin film electrodes, as an example.

The first recordings of neuronal action potentials using substrate integrated, thin film electrodes were made as early as 1977 (Gross, et al. 1977). Subsequent research has led to multi-channel investigations of network dynamics and their applications. Indium-tin oxide was introduced later as a viable microelectrode material and was designed and tested for recording in life support chambers (Gross and Schwalm, 1995). These networks were used to explore stimulation of networks through the recording electrodes (Gross et al., 1994).

Linked dual, age-matched neuronal networks have been grown on microelectrode arrays with for possible uses as biosensors (Gross et al., 1995). A practical and realistic use of neural networks is in their application as physiological function deficit detectors. Due to electrophysiological mechanisms, neurons represent efficient transducers for detecting and recording the dynamics of cell death, receptor-ligand interactions, alterations in metabolism, cell signal transduction cascade events, and generic membrane perforation processes. As such, mammalian networks in culture, devoid of extra-neuronal homeostatic protection mechanisms, function as reliable and highly sensitive detectors of any toxicant capable of interfering with autonomic life support, neuromuscular functions, and even behavior.

Although single neurons are often vulnerable and unreliable, networks of neurons may be used to form robust, fault-tolerant, spontaneously active dynamic systems with high sensitivity to their chemical environment. Networks in culture generate response profiles that are concentration and substance specific and react to a broad range of compounds. Pharmacologically and toxicologically, neuronal networks are representative of the parent tissue.

### SUMMARY OF THE INVENTION

The present invention provides a system for testing the neuronal effects of a compound includes a microelectrode array, a data capture unit communicably coupled to the microelectrode array, a processor communicably coupled to the data capture unit and one or more input/output devices communicably coupled to the processor. The microelectrode array, which can be a MEA detector, is capable of supporting genetically modified neuronal cells and measuring neuronal activity. The microelectrode array can also be a chamber having a fluid input connected to a perfusion system. The processor, which can be a computer, compares the neuronal activity of the genetically modified neuronal cells 104 in the presence and absence of the compound.

The system may also include a first and second chamber in fluid communication, wherein the first chamber is separated from the second chamber by a barrier that acts as a blood-brain barrier. The first chamber can be the microelectrode array 102. The neuronal cells can be from a 12-16 day old embryo, a heterozygous animal, or selected from Tables I, II or III. The neuronal cells can also be selected from the frontal cortex, the auditory cortex, the visual cortex, the hippocampus or the spinal cord. Furthermore, the neuronal cells may include one or more types of neuronal cells, e.g., that can form a neural tissue.

In addition, the present invention provides a method of determining the effects of a sample on genetically modified neuronal cells in accordance with the present invention is shown. A culture of genetically modified neuronal cells is grown on a microelectrode array. A portion of the genetically modified neuronal cells is then exposed to a sample. The effects of the sample on the genetically modified neuronal cells exposed to the sample are measured to determine the effects of the sample on the genetically modified neuronal cells.

The present invention also provides a method of determining the effects of a sample on a neuronal cell in accordance with the present invention is shown. A first neuronal cell from a control animal in grown on a first microelectrode. A second neuronal cell from a homozygous genetically modified animal is grown on a second microelectrode. The first and second neuronal cells are then exposed to a compound. The effects of the compound on the first neuronal cell with the first microelectrode and the second neuronal cell with the second microelectrode are measured. The measurements from the first and the second microelectrode are compared to determine the effects of the compound on the neuronal cell.

### BRIEF DESCRIPTION OF THE DRAWINGS

For a more complete understanding of the features and advantages of the present invention, reference is now made to the detailed description of the invention along with the accompanying figures in which corresponding numerals in the different figures refer to corresponding parts and in which:

FIG. 1 is a block diagram of a system in accordance with the present invention;

FIGS. 2A, 2B, 3A and 3B illustrate typical microelectrode arrays that can be used in connection with the present invention;

FIG. 4 is a flow chart illustrating a method of determining the effects of a sample on genetically modified neuronal cells in accordance with the present invention;

FIG. 5 is a flow chart illustrating a method of determining the effects of a sample on a neuronal cell in accordance with the present invention;

FIG. 6 is a flow chart outlining the basic steps in the cell culturing phase of the testing procedure;

FIG. 7 is a flow chart describing the procedure to prepare the cell culture medium;

FIG. 8 is a flow chart describing the procedure to prepare the dissecting buffer;

FIG. 9 is a flow chart describing the procedure to prepare the other solutions (cell adhesion and enzyme solutions);

FIG. 10 is a flow chart describing the procedure to create the microelectrode array (MEA) substrate;

FIG. 11 is a flow chart describing the procedure to create the electrodes on the MEA substrate;

FIG. 12 is a flow chart describing the procedure to prepare the MEA for cell culturing;

FIG. 13 is a flow chart describing the procedure to determine the cell cultures to be created;

FIG. 14 is a flow chart describing the procedure to prepare a cell culture to be used for the control test culture;

FIG. 15 is a flow chart describing the procedure to prepare a cell culture from a homozygote mating pair to be used for the experimental test culture;

FIG. 16 is a flow chart describing the procedure to prepare a cell culture from a hetrozygote mating pair to be used for the experimental test culture;

FIG. 17 is a flow chart describing the procedure to genotype the cell cultures from a hetrozygote mating pair;

FIG. 18 is a flow chart describing the procedure to nurture and care for the cell cultures;

FIG. 19 is a flow chart outlining the basic steps in the neuroactivity testing procedure;

FIG. 20 is a flow chart describing the procedure to autoclave the testing chamber;

FIG. 21 is a flow chart describing the procedure to select the cell culture to be used for testing;

FIG. 22 is a flow chart describing the procedure to assemble the testing chamber;

FIG. 23 is a flow chart describing the procedure to set up the testing station;

FIG. 24 is a flow chart describing the procedure to set up the testing software;

FIG. 25 is a flow chart describing the procedure to record the reference activity;

FIG. 26 is a flow chart describing the procedure to perform the testing needed for the neuroactivity comparison;

FIG. 27 is a flow chart describing the procedure to analyze the neuroactivity comparison data;

FIG. 28 is a flow chart describing the procedure to use the data to determine if a compound targets the products of a specific gene;

FIG. 29 is a flow chart outlining the basic steps in the dose response analysis testing procedure;

FIG. 30 is a flow chart describing the procedure to perform the testing needed for the dose response curve development;

FIG. 31 is a flow chart describing the procedure to add compounds to the test cultures in the testing chamber; and

FIG. 32 is a flow chart describing the procedure to analyze the dose response data and determine the EC 50.

### DETAILED DESCRIPTION OF THE INVENTION

While the production and application of various embodiments of the present invention are discussed in detail below, it should be appreciated that the present invention provides many applicable inventive concepts that may be embodied in a wide variety of specific contexts. The specific embodiments discussed herein are merely illustrative of specific ways to make and use the invention and do not delimit the scope of the invention.

The present invention takes advantage of mammalian neuronal networks grown on substrate integrated microelectrode arrays (MEAs). Primary cultures from dissociated tissue have superior adhesion to the recording substrate, stability during recording, longevity, and number of active channels that can be observed on the spike level with good signal-to-noise ratios. These observations include (1) neuronal networks most likely respond to ANY substance that has a major effect on central nervous system functions, (2) the sensitivities and efficacies are comparable to those causing responses in vivo, (3) false positives and false negatives appear minimal and, in many cases, may be predictable, (4) agent response profiles are reproducible and, with improved data processing, may identify mechanisms and classify an increasing number of substances, and (5) a simple, reliable warning system can be constructed.

Neuronal Network Biosensors (NNBS) are living nerve cell networks growing on arrays of substrate integrated miroelectrodes in cell culture. The networks are constantly spontaneously active and allow long-term (months) monitoring of action potential (AP or “spike”) patterns from as many as 64 channels simultaneously. These networks, as isolated neural tissue, devoid of the blood-brain barrier and other non-neuronal homeostatic mechanisms are highly sensitive to their environment and respond to chemical and physical changes in the life support medium with increases, decreases, or pattern changes in their spike activity. In addition, AP amplitude decreases reflect metabolic changes that lead to a reduction of the membrane potential.

The readout from such systems is any change from the normal activity that a particular culture has established. Not all networks have identical starting (or native) activity as long as they are spontaneously active. Note that the NNBS does not have to generate exactly the same patterns as the tissue in vivo. It is only necessary to establish a “cultured network correlate response” that can be reliably elicited from networks in response to a certain class of compounds for which the physiological effect is known. For high-throughput application, large numbers of integrated microculture chambers containing a variety of neural and non-neural tissues with a microfluidic system that can mimic normal physiological routing and interactions may be developed.

The NNBS is a generic sensor that mimics pharmacologically the nervous system of an animal. For example disinhibitory compounds all enhance bursting and regularization of the burst pattern. Such compounds all cause epilepsy in mammals. Therefore regularization of burst patterns in cultures and epilepsy may be correlated.

Microelectrode arrays (MEAs) come in single and dual network designs. The dual networks provide a control culture that can monitor the life support system or provide a second network. Use of a dual network array allows the growth of “twin networks” that have the same seeding date, seeding pool, and feeding manipulations. Cultures grown on the dual network array grow under the same medium in isolated adhesion areas and are separated into separate medium pools only upon assembly of the chamber. A dual network design may use a 5×5 cm plate and edge contact arrangement. Each network is served by, e.g., 32 microelectrodes.

Burst pattern changes in response to an agent may be recorded as integrated spike data displayed on a chart recorder. The results from different studies may be recorded and catalogued such that the molecular signature of such agent(s) may be used in sampling unknowns. Examples of compounds that may be tested and catalogued include, e.g mind-altering drugs such as the cannabinoids or even substances that have subtle effects generally detected as tinnitus, hallucinations, vertigo, irritability, loss of concentration, and minor loss of muscle coordination.

Generally, networks with 1,000 to 5,000 neurons growing adhesion areas with 3 to 4 mm diameters may be used. These systems can lose a significant percentage of neurons without showing any deficit in their spontaneous activity or their pharmacological responses.

In operation, neuronal cells over the recording matrix (1 mm² area) and axons from outside the recording matrix supply the spontaneous activity. Despite density fluctuations, a stabilization of neuronal counts past 30 days is obtained. Neuronal losses are approximately 20% in 100 days (6% per month). Neuronal counts include the total number of active signals recorded from the culture. The exclusion of a signal from the count does not signify neuronal cell death, only a loss of activity. NNBS responses are generally histiotypic, that is, the networks act as physiological sensors that can predict the effects of unknown compounds on the nervous system and allow an extrapolation to behavioral deficits.

Furthermore, because the networks express the same receptors and channels found in the parent tissue they have been found to respond very much like the nervous system of an animal would respond. Networks growing in culture on substrate integrated microelectrode arrays serve to link the molecular biochemistry of the network with results from whole animal physiology. The networks of the present invention may be used to provide rapid and accurate information on one or more pharmacological or toxicological changes.

Although a typical network has between 1,000 and 5,000 neurons, the number of inputs in, e.g., a 64-amplifier recording system limit analysis to 64 sites of the network. Using spike separation, e.g., it is possible to record from more than 100 individual neurons, as many electrodes carry signals from more than one axon. With the present 32 DSPs (digital signal processors), 32 channels may be selected for digitizing. Under optimal separation conditions, a user may record a maximum of 128 active units (4×32). For most sensing uses, however, such a high number of channels is more than sufficient.

Responses to toxicants are usually global, i.e., all channels are affected in a highly similar manner. Such responses can be detected reliably (and be quantified) with data from 10 to 20 channels. Responses to hallucinogens may be more complex by generating unit-specific responses where groups of different neurons respond differently. Therefore, the number of electrodes required to give a statistically sound representation of the network depends on the complexity of the response. Fortunately, in toxicology the end points of many, if not most, responses are relatively simple.

Response Quantification. Response quantification occurs generally in three stages:

(1) detection, (2) classification and (3) identification. Detection will depend on independent multivariate z-scores, i.e. on changes of any activity variable or group of activity variables that exceed 2 or 3 standard deviations of the reference activity. Classification is based on simple, but major physiological responses that will be identified as inhibitory, disinhibitory, and excitatory. Whereas inhibition and excitation depend heavily on spike rate, disinhibition (which emerges during generation of epileptiform activity) requires measurement of pattern regularity. An interesting distinction between excitation and disinhibition is that both types of responses increase spike production, however, the resulting patterns are radically different. Excitation increases activity without favoring regularity. Disinhibition (substances that silence inhibitory circuits by blocking GABA and or glycine receptors) always generates bursting and high burst pattern regularity.

Identification after classification is a complex task and requires extensive scrutiny of response profiles and application of a variety of methods that have not yet been completely identified. Response profile matching with those generated by known compounds is certainly an essential step. Using the present invention, a number of systems may be tested and quantified for detection, classified and identified. Often, a single unique feature of the profile may identify a compound, e.g, botulinum toxin A. The features of a Toxin A response includes a long, concentration-independent delay and slow, but irreversible decline of all activity that is highly unique. The delay is caused primarily by receptor dependent internalization of this large protein proenzyme.

Biostatistics. A Plexon MAP 64 channel workstations using Plexon data acquisition software and the NEX Technologies Neuroexplorer program may be used for data acquisition and analysis. The Plexon system allows action potential (AP or spike) discrimination with 32 digital signal processors that simplify the data before it reaches the host computer. In optimal cases, four different active units could be distinguished per channel resulting in a maximum capacity of 128 logical channels available for analysis.

Normally, the 64 electrode MEA yields an average of 30 channels with good signal-to-noise ratios where at least one or two units can be clearly identified and separated on each channel. The 64 electrode MEA yields an operational maximum of 60 logical channels. Both spike time stamps and waveforms may be collected for analyses of pattern changes and influences on membrane potentials or voltage-gated channel performance that would alter the AP wave shape. Data can be exported to Excel, Kaleidagraph, and Matlab (among many other programs for plotting or further statistical analyses).

The multichannel environment is still somewhat unique in electrophysiology and effective methods for optimal network analyses are evolving. The following basic montage of plots for characterization of the network dynamics may be used: (1) temporal evolution of burst and spike rates in terms of cross channel means and their standard deviations; (2) dose-response curves based on spike production on all channels; (3) temporal evolution of burst variables (a) duration, (b) period, (c) max spike frequencies in bursts, and (d) burst coordination across channels. Because studies can last anywhere from 15 minutes to more than 48 hrs and network responses need to be followed in real-time, it is convenient to form “minute means (MM)” for all burst variables (except rate, which is a scalar) and follow the network responses in terms of one minute steps. These minute means are grouped into “experimental episode means (EEM)” that are then compared to the reference activity mean.

The system is often adjusted for substance-specific effects that can influence the final analysis. Often it is necessary to select a “response stationarity” for best results. For example, synaptic receptor-mediated responses are generally rapid, but often decay as the network adapts or as the substance is degraded enzymatically. Conversely, metabotropic receptor-mediated effects are generally slower in changing network activity, but will reach a maximum effect for a variable period of time. In addition, response times are concentration-dependent. Therefore, in this environment, a fixed time protocol must be supplemented by selecting periods of network stationarity, where activity establishes a constant pattern. Therefore “experimental episode means” may be calculated from time periods that are shorter than the episode defined by test substance application to the next medium change.

Networks Statistics. The classical spike train statistics of NEX may be supplemented with more useful network statistics. For example, by using minute means that lead to test episode means, and subsequently cross channel (or network) episode means, and the use of coefficients of variation.

Chip Design. MEAs may be fabricated using, e.g., chromium masks and may be obtained from Photronix, Colorado Springs, Colo. Further customization may be useful for specific applications. MEAs are made often from a rugged glass carrier plate, indium-tin oxide conductors with gold deposits at exposed sites and dimethyl polysiloxane as insulator. MEAs have been found to have a lifetime of several years and are not toxic. MEAs are remarkably rugged, some have been used for 8-10 cycles of use, e.g., 2 months under warm medium for each cycle, followed by autoclaving and flaming to activate the surface before decoration with polylysine and laminin, without an appreciable loss of function.

Sample Collection and Preparation. A generic sensor may be designed and used that has the capability to sample water, air (with appropriate concentration and elution steps), and even human serum and urine. The NNBS is combined with a sample and a 2× concentrations of supply medium in order to obtain a maximum concentration of a potential toxicant. It may even be feasible to obtain a 25% medium, 75% sample water ratio or even higher concentrations of media depending on the solubility of the basic components of the media and their interaction with the sample.

Flow Rates. Closed chambers often operate at 20 to 40 μl per min. This flow rate is dictated by the small laminar flow chamber design that has only a 300 μm space between the cells and the glass window. Higher rates cause shear stress of cells, channel destabilization and changes in activity. Over long periods of time the shear stress will promote Ca⁺⁺ entry and cell death. As these flow rates are too slow for rapid sample detection, the chambers may be modified to accommodate a flow rate of 1 ml per min. If tubing distances are kept to a minimum (such as 20 cm between sample stores and network and small inner diameter tubing is used (1 mm), then a flow rate of 1 ml/min translates to a sampling time of approximately 38 sec.

In operation, the following conditions may be used in a chamber for use with the present invention, namely:

| | |
|---|---|
| Medium Supply: | 200 ml (2X concentration) |
| Internal Water Supply: | 200 ml |
| Total Medium Supply: | 400 ml |

(A) Flow rate through recording chamber at 20-40 μl/min (2.4 ml/hr)

Total Running Time with medium voided: 181 hrs (7.5 days)

Total Running Time (at 40 μl/min) with medium recirculation at a medium usage (voided) of 10 ml/week: 40 weeks (10 months)

(B) Flow rate of 1 ml/min (in modified chambers)

Total Running Time with medium voided: 400 min

Total Running Time with medium recirculation (10 ml per week used & voided): 40 weeks

The above conditions may or may not take sampling into consideration. For example, samples with potential toxic substances are best avoided prior to sampling. Test samples, however, often need to be circulated for a minimum of about 30-360 min. These parameters may be varied depending on the detection time required for pattern stabilization, classification, and possibly identification.

Constant Bath. It is also possible to perform testing with a constant bath chamber. Medium is placed in the chamber (1 ml or 2 ml, depending on the chamber design). Compound aliquots are added in quantities less than 10 μl, giving whole bath compound concentrations in the pico- to micro-range.

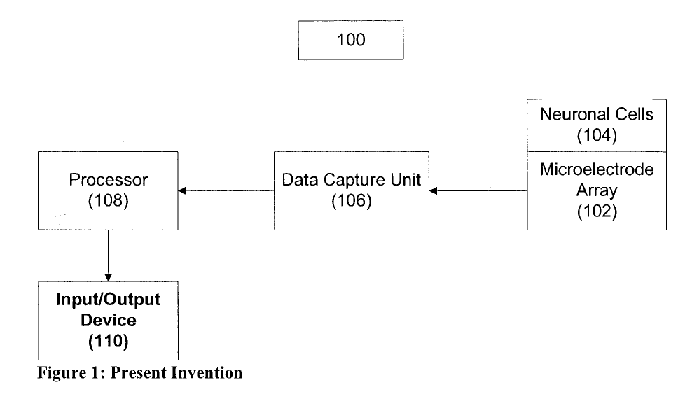

Now referring to FIG. 1, a block diagram of a system 100 in accordance with the present invention is shown. The system 100 for testing the neuronal effects of a compound includes a microelectrode array 102, a data capture unit 106 communicably coupled to the microelectrode array 102, a processor 108 communicably coupled to the data capture unit 106 and one or more input/output devices 110 communicably coupled to the processor 108. The microelectrode array 102, which can be a MEA detector, is capable of supporting genetically modified neuronal cells 104 and measuring neuronal activity. The microelectrode array 102 can also be a chamber having a fluid input connected to a perfusion system. The processor, which can be a computer, compares the neuronal activity of the genetically modified neuronal cells 104 in the presence and absence of the compound.

The system may also include a first and second chamber in fluid communication, wherein the first chamber is separated from the second chamber by a barrier that acts as a blood-brain barrier. The first chamber can be the microelectrode array 102. The neuronal cells can be from a 12-16 day old embryo, a heterozygous animal, or selected from Tables 1 through 3. The neuronal cells can also be selected from the frontal cortex, the auditory cortex, the visual cortex, the hippocampus or the spinal cord. Furthermore, the neuronal cells may include one or more types of neuronal cells. In addition, the neuronal cells can form a neural tissue.

TABLE 1
Sample List of Knockout Mutants

| | | MGI Assession | Neurobiology | | |
|---|---|---|---|---|---|
| Symbol | Symbol Name | ID | Defect | Supplier | Strain Name |
| Apoe | apolipoprotein E | MGI: 88057 | Behavioral & | JAX″ | B6.129-Apoetm1Unc Ldlrtm1Her |
| | | | Learning | | |
| Apoe | apolipoprotein E | MGI: 88057 | Behavioral & | JAX″ | B6.129P2-Apoetm1Unc |
| | | | Learning | | |
| Atm | ataxia telangiectasia | MGI: 107202 | Ataxia | JAX″ | 129S6/SvEvTac-Atmtm1Awb |
| | mutated homolog | | | | |
| | (human) | | | | |
| Cln6 | ceroid-lipofuscinosis, | MGI: 2159324 | Ataxia | JAX″ | B6.Cg-Cln6nclf |
| | neuronal 6 | | | | |
| Dab1 | disabled homolog 1 | MGI: 108554 | Ataxia | JAX″ | CBy.129S4-Dab1tm1Cpr |
| | (Drosophila) | | | | |
| Dab1 | disabled homolog 1 | MGI: 108554 | Ataxia | JAX″ | MRL.129P2(B6)- |
| | (Drosophila) | | | | B2mtm1Unc/Dcr-Dab1scm-2J/+ |
| Dab1 | disabled homolog 1 | MGI: 108554 | Ataxia | JAX″ | STOCK A/A-Dab1scm/+ |
| | (Drosophila) | | | | |
| Gfap | glial fibrillary acidic | MGI: 95697 | Astrocyte | JAX″ | B6; 129S-Gfaptm1Mes |
| | protein | | | | |
| HD | Huntington disease (Human) | | Behavioral & | JAX″ | B6C3F1/J-TgN(HD82Gln)81Dbo |
| | | | Learning | | |
| HD | Huntington disease (Human) | | Ataxia | JAX″ | B6CBA-TgN(HDexon1)61Gpb |
| HD | Huntington disease (Human) | | Ataxia | JAX″ | B6CBA-TgN(HDexon1)62Gpb |
| Hprt | hypoxanthine guanine | MGI: 96217 | Neurodegeneration | JAX″ | B6.129P2-Hprtb-m3 |
| | phosphoribosyl | | | | |
| | transferase | | | | |
| Ldlr | low density lipoprotein | MGI: 96765 | Behavioral & | JAX″ | B6.129-Apoetm1Unc Ldlrtm1Her |
| | receptor | | Learning | | |
| Reln | reelin | MGI: 103022 | Ataxia | JAX″ | B6C3Fe a/a-Relnrl/+ |
| S100b | S100 protein, beta | MGI: 98217 | Astrocyte | JAX″ | C3Sn.BLiA-Pde6b+/Dn- |
| | polypeptide, neural | | | | TgN(S100b)5.12Rhr |
| S100b | S100 protein, beta | MGI: 98218 | Astrocyte | JAX″ | C57BL/6J-TgN(S100b)5.12Rhr |
| | polypeptide, neural | | | | |
| SOD1 | superoxide dismutase 1, soluble (Human) | | Behavioral & | JAX″ | C57BL/6-TgN(SOD1)3Cje |
| | | | Learning | | |
| Agtpbp1 | ATP/GTP binding protein 1 | MGI: 2159437 | Ataxia | JAX″ | B6.BR-Agtpbp1pcd |
| Agtpbp1 | ATP/GTP binding protein 1 | MGI: 2159437 | Ataxia | JAX″ | B6C3Fe a/a-Agtpbp1pcd/+ |
| Agtpbp1 | ATP/GTP binding protein 1 | MGI: 2159437 | Ataxia | JAX″ | BALB/cByJ-Agtpbp1pcd-3J |
| anx | anorexia | MGI: 88029 | Behavioral & | JAX″ | B6C3Fe a/a-anx |
| | | | Learning | | |
| Atp2b2 | ATPase, Ca++ | MGI: 105368 | Ataxia | JAX″ | C3H/HeJ-Atp2b2dfw/+ |
| | transporting, plasma | | | | |
| | membrane 2 | | | | |
| Atp2b2 | ATPase, Ca++ | MGI: 105368 | Ataxia | JAX″ | C57BL-Atp2b2dfw-3J/+ |
| | transporting, plasma | | | | |
| | membrane 2 | | | | |
| Atp2b2 | ATPase, Ca++ | MGI: 105368 | Ataxia | JAX″ | CBy.A-fsn+? Atp2b2dfw-2J |
| | transporting, plasma | | | | |
| | membrane 2 | | | | |
| Atp7a | ATPase, Cu++ | MGI: 99400 | Ataxia | JAX″ | B6.Cg-Atp7aMo-blo |
| | transporting, alpha | | | | |
| | polypeptide | | | | |
| Atp7a | ATPase, Cu++ | MGI: 99400 | Ataxia | JAX″ | B6Ei.Cg-Atp7aMo-blo |
| | transporting, alpha | | | | |
| | polypeptide | | | | |
| ax | ataxia | MGI: 88124 | Ataxia | JAX″ | B6.Cg-axJ |
| bc | bouncy | MGI: 88134 | Ataxia | JAX″ | C3H/HeSn-bc3J/+ |
| Bmp5 | bone morphogenetic | MGI: 88181 | Ataxia | JAX″ | B6.Cg-Rorasg++/+Myo5ad |
| | protein 5 | | | | Bmp5se |
| Ca | caracul | MGI: 88238 | Ataxia | JAX″ | B6C3Fe a/a-Ca Scn8amed-J |
| Cacna1a | calcium channel, | MGI: 109482 | Ataxia | JAX″ | B6.Cg-Os+/+Cacna1atg-la |
| | voltage-dependent, P/Q | | | | |
| | type, alpha 1A subunit | | | | |
| Cacna1a | calcium channel, voltage-dependent, P/Q | | Ataxia | JAX″ | B6.D2-Cacna1atg |
| | type, alpha 1A subunit | | | | |
| Cacna2d2 | calcium channel, | MGI: 1929813 | Ataxia | JAX″ | TKDU/Dn |
| | voltage-dependent, | | | | |
| | alpha 2/delta subunit 2 | | | | |
| Cacnb4 | calcium channel, | MGI: 103301 | Ataxia | JAX″ | B6EiC3Sn a/A-Cacnb4lh |
| | voltage-dependent, beta | | | | |
| | 4 subunit | | | | |
| Cacng2 | calcium channel, | MGI: 1316660 | Ataxia | JAX″ | B6C3Fe a/a-Cacng2stg/+ |
| | voltage-dependent, | | | | |
| | gamma subunit 2 | | | | |
| Cacng2 | calcium channel, | MGI: 1316660 | Ataxia | JAX″ | C57BL/6J-Cacng2stg-wag |
| | voltage-dependent, | | | | |
| | gamma subunit 2 | | | | |
| Calb1 | calbindin-28K | MGI: 88248 | Ataxia | JAX″ | B6.129-Calb1tm1Mpin |
| Catna2 | catenin alpha 2 | MGI: 88275 | Ataxia | JAX″ | C3H/HeSnJ-Catna2cdf/+ |
| Cln6 | ceroid-lipofuscinosis, | MGI :2159324 | Ataxia | JAX″ | STOCK a/aCln6nclf |
| | neuronal 6 | | | | |
| Cstb | cystatin B | MGI: 109514 | Ataxia | JAX″ | 129-Cstbtm1Rm |
| enr | enervated | MGI: 1196289 | Ataxia | JAX″ | B6.Cg-enrTg36Pop |
| Es10 | esterase 10 | MGI: 95421 | Metabolism | JAX″ | WLHR/Le |
| Espn | espin | MGI: 1861630 | Ataxia | JAX″ | JE/Le |
| Foxg1 | forkhead box G1 | | Astrocyte | JAX″ | 129.Cg-Foxg1tm1(cre)Skm |
| Fyn | Fyn proto-oncogene | MGI: 95602 | Behavioral & | JAX″ | 129-Fyntm1Sor |
| | | | Learning | | |
| Gnb5 | guanine nucleotide | MGI: 101848 | Ataxia | JAX″ | B6 × B6CBCa Aw-J/A-Myo5aflr |
| | binding protein, beta 5 | | | | Gnb5flr |
| gnd | generalized neuroaxonal | MGI: 95786 | Ataxia | JAX″ | C3HeB/FeJLe a/a-gnd |
| | dystrophy | | | | |
| gr | grizzled | MGI: 95803 | Ataxia | JAX″ | JIGR/Dn |
| Grid2 | glutamate receptor, | MGI: 95813 | Ataxia | JAX″ | B6 × BALB/cByJ-Grid2Lc-J/+ |
| | ionotropic, delta 2 | | | | |
| Grid2 | glutamate receptor, | MGI: 95813 | Ataxia | JAX″ | B6CBACa Aw-J/A-Grid2Lc |
| | ionotropic, delta 2 | | | | |
| Grid2 | glutamate receptor, | MGI: 95813 | Ataxia | JAX″ | C57BL/6J-Grid2ho-5J/+ |
| | ionotropic, delta 2 | | | | |
| Grid2 | glutamate receptor, | MGI: 95813 | Ataxia | JAX″ | DBA/2J-Grid2ho-4J/+ |
| | ionotropic, delta 2 | | | | |
| Herc2 | hect (homologous to the | MGI: 103234 | Ataxia | JAX″ | BALB/cByJ-Herc2J/+ |
| | E6-AP (UBE3A) carboxyl | | | | |
| | terminus) domain and | | | | |
| | RCC1 (CHC1)-like | | | | |
| | domain (RLD) 2 | | | | |
| hop | hop-sterile | MGI: 96168 | Ataxia | JAX″ | STOCK hop/+ |
| Hq | harlequin | MGI: 96222 | Ataxia | JAX″ | B6CBACa-Aw-J/A-Hq |
| hr | hairless | MGI: 96223 | Cancer Research | JAX″ | WLHR/Le |
| hyh | hydrocephaly with hop | MGI: 96302 | Ataxia | JAX″ | B6C3Fe-a/a-hyh |
| | gait | | | | |
| ji | jittery | MGI: 96640 | Ataxia | JAX″ | C3HeB/FeJ-jihes/+ |
| ji | jittery | MGI: 96640 | Ataxia | JAX″ | JIGR/Dn |
| Kcna1 | potassium voltage-gated | MGI: 96654 | Ataxia | JAX″ | C3HeB.129S7(B6)- |
| | channel, shaker-related | | | | Kcna1tm1Tem |
| | subfamily, member 1 | | | | |
| Kcnj6 | potassium inwardly- | MGI: 104781 | Ataxia | JAX″ | B6CBACa Aw-J/A-Kcnj6wv |
| | rectifying channel, | | | | |
| | subfamily J, member 6 | | | | |
| Klc1 | kinesin light chain 1 | | Ataxia | JAX″ | B6.129S-Klc1tm1Gsn |
| lacZ | beta-galactosidase | | Astrocyte | JAX″ | FVB/NJ-TgN(XGFAP-lacZ)3Mes |
| Lepr | leptin receptor | MGI: 104993 | Ataxia | JAX″ | BKS.Cg-meaJ Leprdb+/++m |
| Lpin1 | lipin 1 | MGI: 1891340 | Ataxia | JAX″ | B6 × BALB/cByJ-Lpin1fld/+ |
| Lpin1 | lipin 1 | MGI: 1891340 | Ataxia | JAX″ | BALB/cByJ-Lpin1fld/+ |
| Lpin1 | lipin 1 | MGI :1891340 | Ataxia | JAX″ | C3H/HeJ-Lpin1fld-2J/+ |
| MbpReg | myelin basic protein regulatory region | | Ataxia | JAX″ | B6.Cg-enrTg36Pop |
| mdf | muscle deficient | MGI: 96948 | Ataxia | JAX″ | B6C3Fe a/a-mdf |
| mea | meander tail | MGI: 96956 | Ataxia | JAX″ | BKS.Cg-mea2J m/++ |
| mea | meander tail | MGI: 96956 | Ataxia | JAX″ | BKS.Cg-meaJ Leprdb +/++m |
| mnd | motor neuron | MGI: 97038 | Ataxia | JAX″ | AK.B6-mnd/+ |
| | degeneration | | | | |
| mnd | motor neuron | MGI: 97038 | Ataxia | JAX″ | B6.KB2-mnd/MsrJ |
| | degeneration | | | | |
| Myo5a | myosin Va | MGI: 105976 | Ataxia | JAX″ | B6 × B6CBCa Aw-J/A-Myo5aflr |
| | | | | | Gnb5flr |
| Myo5a | myosin Va | MGI: 105976 | Ataxia | JAX″ | B6.Cg-Rorasg++/+Myo5ad |
| | | | | | Bmp5se |
| Npc1 | Niemann Pick type C1 | MGI: 1097712 | Ataxia | JAX″ | C57BLKS/J-Npc1spm/+ |
| Npc1N | NIH allele | | Ataxia | JAX″ | BALB/cNctr-Npc1N/+ |
| nr | nervous | MGI: 97375 | Ataxia | JAX″ | C3Fe.CGr-nr |
| Ntf3 | neurotrophin 3 | MGI: 97380 | Ataxia | JAX″ | B6.129S4-Ntf3tm1Jae |
| Ntf3 | neurotrophin 3 | MGI: 97380 | Ataxia | JAX″ | STOCK Ntf3tm1Jae |
| Ntf4 | neurotrophin 4 | MGI: 97381 | Ataxia | JAX″ | B6.129S4-Ntf3tm2Jae |
| Re | rex | MGI: 97888 | Ataxia | JAX″ | SHR/GnEi |
| Rora | RAR-related orphan | MGI: 104661 | Ataxia | JAX″ | B6.Cg-Rorasg++/+Myo5ad |
| | receptor alpha | | | | Bmp5se |
| Rora | RAR-related orphan | MGI: 104661 | Ataxia | JAX″ | B6.Cg-Rorasg/+ |
| | receptor alpha | | | | |
| Rora | RAR-related orphan | MGI: 104661 | Ataxia | JAX″ | B6C3Fe a/a-Rorasg/+ |
| | receptor alpha | | | | |
| SCA2 | spinocerebellar ataxia 2 | | Ataxia | JAX″ | B6D2-TgN(Pcp2SCA2)11Plt |
| | (olivopontocerebellar ataxia 2, autosomal | | | | |
| | dominant, ataxin 2) (Human) | | | | |
| Scn8a | sodium channel, voltage- | MGI: 103169 | Ataxia | JAX″ | B6C3Fe a/a-Ca Scn8amed-J |
| | gated, type VIII, alpha | | | | |
| | polypeptide | | | | |
| Scn8a | sodium channel, voltage- | MGI: 103169 | Ataxia | JAX″ | C3HeB/FeJ-Scn8amed |
| | gated, type VIII, alpha | | | | |
| | polypeptide | | | | |
| Scn8a | sodium channel, voltage-gated, type VIII, | | Ataxia | JAX″ | C57BL/6J-Scn8amed-jo |
| | alpha polypeptide | | | | |
| Sfxn1 | sideroflexin | MGI: 2137677 | Ataxia | JAX″ | JE/Le |
| shm | shambling | MGI: 98298 | Ataxia | JAX″ | SHR/GnEi |
| shmy | shimmy | MGI: 99514 | Ataxia | JAX″ | STOCK a/a Tyrp1b/Tyrp1b |
| | | | | | shmy/+ |
| Slc9a1 | solute carrier family 9 | MGI: 102462 | Ataxia | JAX″ | B6.SJL-Slc9a1swe/+ |
| | (sodium/hydrogen | | | | |
| | exchanger), member 1 | | | | |
| Slc9a1 | solute carrier family 9 | MGI: 102462 | Ataxia | JAX″ | B6SJLF1-Slc9a1swe/+ |
| | (sodium/hydrogen | | | | |
| | exchanger), member 1 | | | | |
| Slc9a1 | solute carrier family 9 | MGI: 102462 | Ataxia | JAX″ | SJL/J-Slc9a1swe/+ |
| | (sodium/hydrogen | | | | |
| | exchanger), member 1 | | | | |
| Snca | synuclein, alpha | | Astrocyte | JAX″ | B6; 129X1-Sncatm1Rosl |
| Spnb4 | beta-spectrin 4 | MGI: 1890574 | Ataxia | JAX″ | C3FeB6-A/Aw-J-Spnb4qv-J |
| Spnb4 | beta-spectrin 4 | MGI: 1890574 | Ataxia | JAX″ | C3H/HeJ-Spnb4qv-Ind2J/+ |
| Spnb4 | beta-spectrin 4 | MGI: 1890574 | Ataxia | JAX″ | C57BL/6J-Spnb4qv-3J/+ |
| stu | stumbler | MGI: 98439 | Ataxia | JAX″ | B6.C3-stu |
| tip | tippy | MGI: 98757 | Ataxia | JAX″ | B6C3Fe-a/a-tip |
| tk | tail-kinks | MGI: 98760 | Ataxia | JAX″ | TKDU/Dn |
| Unc5h3 | unc5 homolog (C. | MGI: 1095412 | Ataxia | JAX″ | B6C3Fe-a/a-Unc5h3rcm |
| | elegans) 3 | | | | |
| wl | wabbler-lethal | MGI: 98951 | Ataxia | JAX″ | C3H/HeSnJ-wlvmd/+ |
| wl | wabbler-lethal | MGI: 98951 | Ataxia | JAX″ | WLHR/Le |
| Wnt1 | wingless-related MMTV | MGI: 98953 | Ataxia | JAX″ | B6C3Fe-a/a-Wnt1sw |
| | integration site 1 | | | | |

TABLE 2
Tissues and Cells

| Animal | | | Effected | | Culture | |
|---|---|---|---|---|---|---|
| Type | Name | Publication | Tissue | Disease | Type | Description |
| Mouse | Nf1(n31) | Endocr Pathol 1995 | | pheochromocytomas | | |
| | | Winter; 6(4): 323-335 | | | | |
| Mouse | M4 | J Neurosci 2002 Jun | | | | muscarinic M2 acetylcholine receptors |
| | | 15; 22(12): RC229 | | | | Monoamine oxidase-A knockout (MAO- |
| | | | | | | A KO) mice have elevated brain |
| | | | | | | serotonin (5-HT) and noradrenaline |
| | | | | | | (NA) levels, and one would therefore |
| | | | | | | anticipate increased monoamine |
| | | | | | | release and compensatory changes in |
| | | | | | | other aspects of presynaptic |
| | | | | | | monoamine function |
| Mouse | Monoamine | Eur J Neurosci 2002 | | | Slice | We found that p75(−/−) neurons did not |
| | oxidase-A | May; 15(9): 1516-22 | | | | release acetylcholine in response to |
| | | | | | | BDNF and that neurons overexpressing |
| | | | | | | p75 showed increased cholinergic |
| | | | | | | transmission, indicating that the actions |
| | | | | | | of BDNF are mediated through the p75 |
| | | | | | | neurotrophin receptor. |
| Rodent | p75 | Nat Neurosci 2002 | | | | Rag1-knockout (Rag1−/−) mice, which |
| | | Jun; 5(6): 539-45 | | | | lack mature B and T lymphocytes |
| Mouse | Rag-1 | Science 2002 May | | | | Our findings indicate a role for the SDF- |
| | | 3; 296(5569): 927-31 | | | | 1/CXCR4 chemokine signaling system |
| | | | | | | in DG morphogenesis. Finally, the DG |
| | | | | | | is unusual as a site of adult |
| | | | | | | neurogenesis. We find that both CXCR4 |
| | | | | | | and SDF-1 are expressed in the adult |
| | | | | | | DG, suggesting an ongoing role in DG |
| | | | | | | morphogenesis |
| Mouse | CXCR4 0 | Proc Natl Acad Sci | | | | we identify Id2 as an induced gene |
| | | USA 2002 May 14; | | | | during serum and potassium |
| | | 99(10): 7090-5 | | | | deprivation-induced apoptosis of |
| | | | | | | cerebellar granule neurons |
| Mouse | Id2 | J Neurochem 2002 | | | | Fast spiking (FS), GABAergic neurons |
| | | Mar; 80(5): 755-62 | | | | of the reticular thalamic nucleus (RTN) |
| | | | | | | are capable of firing high-frequency |
| | | | | | | trains of brief action potentials, with little |
| | | | | | | adaptation. Studies in recombinant |
| | | | | | | systems have shown that high-voltage- |
| | | | | | | activated K(+) channels containing the |
| | | | | | | Kv3.1 and/or Kv3.2 subunits display |
| | | | | | | biophysical properties that may |
| | | | | | | contribute to the FS phenotype |
| Mouse | Kv3.1 0 | J Neurophysiol 2002 | | | | We conclude that environmental levels |
| | | Mar; 87(3): 1303-10 | | | | of bFGF regulate neonatal hippocampal |
| | | | | | | neurogenesis. |
| Mouse | bFGF 0 | Eur J Neurosci 2002 | Hippocapus growth | | | In conclusion, TH-positive neurons may |
| | | Jan; 15(1): 3-12 | | | | be generated in ventral mesencephalic |
| | | | | | | tissue of Nurr1 deficient mice, |
| | | | | | | suggesting that Nurr1 is not required for |
| | | | | | | TH gene expression in ventral midbrain |
| | | | | | | in vitro. |
| Mouse | Nurr1 | Brain Res Dev Brain | | | | |
| | | Res 2002 Jan 31; | | | | |
| | | 133(1): 37-47 | | | | |
| Mouse | Sandoff | Neuropathol Appl | Neurobiol 2002 | Sandhoff disease | | Disease model? |
| | | Feb; 28(1): 23-34 | | | | |
| Mouse | GLT-1 | Eur J Neurosci 2002 | | | Slice | These data suggest a potential |
| | | Jan; 15(2): 308-14 | | | | mechanism whereby apoE4 may play a |
| | | | | | | role in regenerative failure and |
| | | | | | | accelerate the development of AD |
| Mouse | apoE | Brain Res 2002 Feb | | Alzheimer's | | These results demonstrate that chronic |
| | | 22; 928(1-2): 96-105 | | | | reduction of PS1 activity leads to |
| | | | | | | impaired synaptic plasticity, thus |
| | | | | | | suggesting a role for PS1 in normal |
| | | | | | | cognitive function. |
| Mouse | PS1(+/−) | Neurosci Lett 2002 | Hippocapus | | | |
| | | Feb 8; 319(1): 37-40 | | | | |
| Mouse | TGF-beta | Cell Tissue Res 2002 | | | | |
| | | Jan; 307(1): 1-14 | | | | |
| Mouse | Brn3a | Development 2002 | Retina development | | | |
| | | Jan; 129(2): 467-77 | | | | |
| Mouse | Brn3b | Development 2002 | Retina development | | | |
| | | Jan; 129(2): 467-77 | | | | |
| Mouse | Brn3c | Development 2002 | Retina development | | | |
| | | Jan; 129(2): 467-77 | | | | |
| AAV | NaGlu | Mol Ther 2002 | | Mucopolys | Primary | These results provide a basis for the |
| | | Jan; 5(1): 42-9 | | accharidosis | | development of a treatment for |
| | | | | (MPS) | | neurological disease in MPS IIIB |
| | | | | IIIB | | patients using AAV vectors. |
| | | | | | | Reports that apoptosis within |
| | | | | | | populations of neurotrophin-dependent |
| | | | | | | neurones is virtually eliminated BAX- |
| | | | | | | deficient mice and that BAX-deficient |
| | | | | | | neurones survive indefinitely in culture |
| | | | | | | without neurotrophins have led to the |
| | | | | | | view that BAX is required for the death |
| | | | | | | of neurotrophin-deprived neurones |
| Mouse | BAX | Development 2001 | CNS | | | |
| | | Dec; 128(23): 4715-28 | | | | |
| Mouse | TNF 0 | Nat Neurosci 2001 | | | | |
| | | Dec; 4(12): 1194-8 | | | | |
| Mouse | PLCbeta4 | Neuroreport 2001 | Rostral cerebellum | | | Development of oligodendrocytes and |
| | | Sep 17; 12(13): | | | | the generation of myelin internodes |
| | | 2919-22 | | | | within the spinal cord depends on |
| | | | | | | regional signals derived from the |
| | | | | | | notochord and axonally derived signals. |
| Mouse | ErbB2 | J Cell Biol 2001 Sep | Spinal cord | | | |
| | | 17; 154(6): 1245-58 | development | | | |
| Hamster | APP-C59 | J Neurochem 2001 | | | Primary | |
| | | Sep; 78(5): 1168-78 | | | | |
| Mouse | tPA | J Cereb Blood Flow | | | Primary | |
| | | Metab 2001 Jun; | | | | |
| | | 21(6): 631-4 | | | | |
| AAV | AdTRMet | Biochem Biophys Res | prion | | | transmissible spongiform encephalopathies |
| | | Commun 2001 Jul | protein | | | |
| | | 20; 285(3): 623-32 | | | | |
| AAV | AdTRVal | Biochem Biophys Res | prion | | | transmissible spongiform encephalopathies |
| | | Commun 2001 Jul | protein | | | |
| | | 20; 285(3): 623-32 | | | | |
| ? | CHO 1 | Biochem Biophys Res | prion | | | transmissible spongiform encephalopathies |
| | | Commun 2001 Jul | protein | | | |
| | | 20; 285(3): 623-32 | | | | |
| Mouse | apolipoprotein E | Proc Natl Acad Sci | Hippocampus | Alzheimer's | | Cultured hippocampal slices prepared |
| | | USA 2001 Jul | | | | from apolipoprotein E-deficient mice |
| | | 17; 98(15): 8832-7 | | | | were exposed to an inhibitor of |
| | | | | | | cathepsins B and L |
| Mouse | Brn 3.0- | Mech Dev 2001 | | | | In mice lacking Shh, Brn3.0- and Pax7- |
| | | Jul; 105(1-2): 129-45 | | | | expressing neurons typical of the |
| | | | | | | tectum develop throughout the ventral |
| | | | | | | midbrain, and gene expression patterns |
| | | | | | | characteristic of early tegmental |
| | | | | | | development do not appear. |
| Mouse | Pax 7- | Mech Dev 2001 | | | | In mice lacking Shh, Brn3.0- and Pax 7- |
| | | Jul; 105(1-2): 129-46 | | | | expressing neurons typical of the |
| | | | | | | tectum develop throughout the ventral |
| | | | | | | midbrain, and gene expression patterns |
| | | | | | | characteristic of early tegmental |
| | | | | | | development do not appear. |
| Mouse | Shh | Mech Dev 2001 | | | | In mice lacking Shh, Brn3.0- and Pax7- |
| | | Jul; 105(1-2): 129-44 | | | | expressing neurons typical of the |
| | | | | | | tectum develop throughout the ventral |
| | | | | | | midbrain, and gene expression patterns |
| | | | | | | characteristic of early tegmental |
| | | | | | | development do not appear. |
| Mouse | PAs | J Neurosci 2001 Jun | PNS | | | |
| | | 15; 21(12): 4348-55 | | | | |
| Mouse | tPA | J Neurosci 2001 Jun | PNS | | | |
| | | 15; 21(12): 4348-56 | | | | |
| Mouse | uPA | J Neurosci 2001 Jun | PNS | | | |
| | | 15; 21(12): 4348-57 | | | | |
| Mouse | BNDF | J Neurosci 2001 Jun | Dorsal | cell death | | |
| | | 1; 21(11): 3904-10 | thalamus | | | |
| Mouse | MOR −/− | Eur J Neurosci 2001 | brainstem | | | |
| | | May; 13(9): 1703-10 | | | | |
| Mouse | Prnp 0/0 | Neurobiol Dis 2001 | Hippocampus | | | These data provide strong evidence |
| | | Apr; 8(2): 324-30 | | | | that Ca2+-activated K+ currents in |
| | | | | | | Prnp(0/0) mice are reduced due to an |
| | | | | | | alteration of intracellular calcium |
| | | | | | | homeostasis. |
| Mouse | HPRT 0 | Neurosci Lett 2001 | | Lesch-Nyhan | | Lesch-Nyhan syndrome (LNS), caused |
| | | Apr 27; 303(1): 45-8 | | syndrome | | by the complete deficiency of |
| | | | | | | hypoxanthine |
| | | | | | | phosphoribosyltransferase (HPRT), |
| | | | | | | Cultured striatal neurons from p50−/− |
| | | | | | | mice exhibited enhanced oxidative |
| | | | | | | stress, perturbed calcium regulation, |
| | | | | | | and increased cell death following |
| | | | | | | exposure to 3NP, suggesting a direct |
| | | | | | | adverse effect of p50 deficiency in |
| | | | | | | striatal neurons |
| Mouse | p50 subunit of | J Mol Neurosci 2000 | | Huntington's disease | | |
| | NF-kappaB | Aug; 15(1): 31-44 | | | | |
| Mouse | Fyn −/− | J Neurosci Res 2001 | | | | |
| | | Feb 15; 63(4): 303-12 | | | | |
| Mouse | Bax | J Neurochem 2001 | | Glutamate cell death | | |
| | | Jan; 76(1): 295-301 | | | | |
| Mouse | GalNAc-T | Proc Natl Acad Sci | | | | GalNAc-T knockout mice are lacking a |
| | | USA 2001 Jan 2; | | | | calcium regulatory mechanism |
| | | 98(1): 307-12 | | | | |
| Mouse | Ip(A1) | Proc Natl Acad Sci | Olfactory | | | |
| | | USA 2000 Nov | | | | |
| | | 21; 97(24): 13384-9 | | | | |
| Mouse | APLP1 | J Neurosci 2000 Nov | | Alzheimer's | | |
| | | 1; 20(21): 7951-64 | | | | |
| Mouse | APLP2 | J Neurosci 2000 Nov | | Alzheimer's | | |
| | | 1; 20(21): 7951-65 | | | | |
| Mouse | APP | J Neurosci 2000 Nov | | Alzheimer's | | |
| | | 1; 20(21): 7951-63 | | | | |
| Mouse | DCC | Nature 2000 Oct | dorsal | | | |
| | | 12; 407(6805): 747-50 | spinal | | | |
| | | | cord | | | |
| Mouse | netrin-1 | Nature 2000 Oct | dorsal | | | |
| | | 12; 407(6805): 747-50 | spinal | | | |
| | | | cord | | | |
| Mouse | FMRP | Cereb Cortex 2000 | | retardation | | These observations may have important |
| | | Oct; 10(10): 1045-52 | | | | implications for the understanding of |
| | | | | | | mental retardation associated with the |
| | | | | | | absence of FMRP |
| Mouse | bFGF | Endocrinology 2000 | | | | |
| | | Sep; 141(9): 3065-71 | | | | |
| Mouse | IL-10 | Eur J Neurosci 2000 | | brain inflammation | | |
| | | Jul; 12(7): 2265-72 | | | | |
| Mouse | nNOS | Nitric Oxide 2000 | | | | |
| | | Aug; 4(4): 343-53 | | | | |
| Mouse | TrkB | Neuroscience | | | Primary | |
| | | 2000; 98(3): 437-47 | | | | |
| Mouse | Gfra1 | J Neurosci 2000 Jul | Motor | | | |
| | | 1; 20(13): 4992-5000 | neurons | | | |
| Mouse | Gfra2 | J Neurosci 2000 Jul | Motor | | | |
| | | 1; 20(13): 4992-5000 | neurons | | | |
| Mouse | GPx-1 | J Neurochem 2000 | | Parkinson's disease | | |
| | | Jun; 74(6): 2305-14 | | | | |
| Mouse | Bax 0 | J Neurosci 2000 Jun | | leukemia | | |
| | | 1; 20(11): 4198-205 | | | | |
| AAV | Bcl-2 1 | J Neurosci 2000 Jun | | leukemia | | |
| | | 1; 20(11): 4198-205 | | | | |
| Mouse | p75(LNTR | J Neurosci 2000 Jun | | leukemia | | |
| | | 1; 20(11): 4198-205 | | | | |
| Mouse | N-CAM | J Neurosci 2000 May | Hippocampus | | | |
| | | 15; 20(10): 3631-40 | | | | |
| Mouse | p53 | J Neurosci Res 2000 | | | | |
| | | May 15; 60(4): 450-7 | | | | |
| Mouse | WNT-7a | Cell 2000 Mar | Cerebellum | | | |
| | | 3; 100(5): 525-35 | | | | |
| Mouse | IFNAR-2 | J Interferon Cytokine | Res 2000 | Down's Syndrome | | |
| | | Feb; 20(2): 197-203 | | | | |
| Mouse | IFNGR | J Interferon Cytokine | Res 2000 | Down's Syndrome | | |
| | | Feb; 20(2): 197-203 | | | | |
| Mouse | GDNF | Eur J Neurosci 2000 | | | | |
| | | Feb; 12(2): 446-56 | | | | |
| Mouse | LIF | Neurosci Lett 2000 | Lukemia | | | |
| | | Mar 10; 281 (2-3): | | | | |
| | | 107-10 | | | | |
| Mouse | BDNF | J Neurosci Res 2000 | Sensory | | Primary | |
| | | Feb 1; 59(3): 372-8 | Neurons | | culture | |
| Mouse | NCAM | J Neurosci 2000 Feb | Olfactory | | | |
| | | 15; 20(4): 1446-57 | | | | |
| Mouse | PS1 | J Neurosci 2000 Feb | | Alzheimer's | | |
| | | 15; 20(4): 1358-64 | | | | |
| Mouse | GAP-43 | Development 2000 | Retina development | | | |
| | | Mar; 127(5): 969-80 | | | | |
| Mouse | CPP32 | J Neurosci Res 2000 | | cell death | | |
| | | Jan 1; 59(1): 24-31 | | | | |
| Mouse | CB | Brain Res Mol Brain | Hippocampus | | | |
| | | Res 2000 Jan | | | | |
| | | 10; 75(1): 89-95 | CNS | | | |
| Mouse | Hes1 | J Neurosci 2000 Jan | development | | | |
| | | 1; 20(1): 283-93 | | | | |
| Mouse | Nf1 | Ann N Y Acad Sci | | | | |
| | | 1999 Sep | | | | |
| | | 14; 883: 203-14 | | | | |
| Mouse | ApoE | J Neurochem 1999 | | Alzheimer's | | |
| | | Dec; 73(6): 2613-6 | | | | |
| Mouse | geph | J Neurosci 1999 Nov | | | | Thus, gephyrin is required for the synaptic |
| | | 1; 19(21): 9289-97 | | | | localization of GlyRs and GABA(A) |
| | | | | | | receptors containing the gamma2 and/or |
| | | | | | | alpha2 subunits but not for the targeting of |
| | | | | | | these receptors to the |
| | | | | | | neuronal plasma membrane |
| Mouse | p50 subunit of | J Neurosci 1999 Oct | Hippocampus | | | Collectively, the data demonstrate an |
| | NF-kappaB | 15; 19(20): 8856-65 | | | | important role for the p50 subunit of |
| | | | | | | NF-kappaB in protecting neurons against |
| | | | | | | excitotoxic cell death. |
| Mouse | MMP-9 | J Neurosci 1999 Oct | | | | |
| | | 1; 19(19): 8464-75 | | | | |
| Mouse | SOD1 | Am J Pathol 1999 | | | | |
| | | Aug; 155(2): 663-72 | | | | |
| Mouse | Pax6 | Development 1999 | Retina development | | | |
| | | Aug; 126(16): | | | | |
| | | 3585-96 | | | | |
| Mouse | ApoE | Neuroscience | | Alzheimer's | | |
| | | 1999; 91(3): 1009-16 | | | | |
| Mouse | LIF | Neuroscience | | leukemia | | |
| | | 1999; 89(4): 1123-34 | | | | |
| Mouse | Brn-3a | Development 1999 | | | | |
| | | Jul; 126(13): 2869-82 | | | | |
| Mouse | NT-3 | Dev Biol 1999 Jun | Nerve | | | |
| | | 15; 210(2): 411-27 | growth | | | |
| Mouse | BDNF | Eur J Neurosci 1999 | | | | |
| | | May; 11(5): 1567-76 | | | | |
| Mouse | GAD67 | Neurosci Res 1999 | | | | |
| | | Mar; 33(3): 233-7 | | | | |
| Mouse | Bcl-x | Cell Death Differ | | | | |
| | | 1998 Oct; 5(10): | | | | |
| | | 901-10 | | | | |
| Mouse | Agrin | Dev Biol 1999 Jan | | | | |
| | | 1; 205(1): 65-78 | | | | |
| Mouse | MAG | Mol Cell Neurosci | PNS | | | |
| | | 1998 Sep; 12(1-2): | | | | |
| | | 79-91 | | | | |
| Mouse | TrkB | J Neurosci 1998 Sep | Hippocampus | | | |
| | | 15; 18(18): 7336-50 | | | | |
| Mouse | TrkC | J Neurosci 1998 Sep | Hippocampus | | | |
| | major Ca- | 15; 18(18): 7336-51 | | | | |
| | sensitive | | | | | |
| | adenylyl | | | | | |
| | cyclase | | | | | |
| | isoform of | | | | | |
| | cerebellar | | | | | |
| Mouse | cortex (type I) | Neuron 1998 | | | | |
| | | Jun; 20(6): 1199-210 | | | | |
| Mouse | GFAP | Exp Cell Res 1998 | | | | |
| | | Mar 15; 239(2): | | | | |
| | | 332-43 | | | | |
| Mouse | betaPP | J Neurosci 1997 Dec | Hippocampus | | | |
| | | 15; 17(24): 9407-14 | | | | |
| Mouse | TGF-alpha | J Neurosci 1997 Oct | | | | |
| | | 15; 17(20): 7850-9 | | | | |
| Mouse | Prpc | Exp Neurol 1997 | | | | |
| | | Jul; 146(1): 104-12 | | | | |
| Mouse | PrP | Eur J Neurosci 1997 | | Creutzfeldt-Jakob | | |
| | | Jun; 9(6): 1162-9 | | | | |
| Mouse | Blc-2 | Eur J Neurosci 1997 | | | | |
| | | Apr; 9(4): 848-56 | | | | |
| Mouse | Prp | J Neural Transm | | | | |
| | | Suppl 1997; 50: | | | | |
| | | 191-210 | | | | |
| Mouse | MAG | J Neurosci Res 1996 | | | | |
| | | Nov 15; 46(4): 404-14 | | | | |
| Mouse | BDNF | J Neurosci 1996 Sep | | | | |
| | | 1; 16(17): 5361-71 | | | | |
| Mouse | NT4 | J Neurosci 1996 Sep | | | | |
| | | 1; 16(17): 5361-72 | | | | |
| Mouse | p53 | J Cell Sci 1996 | | | | |
| | | Jun; 109 (Pt 6): | | | | |
| | | 1509-16 | | | | |
| Mouse | CNTFR | Mol Cell Neurosci | | | | |
| | | 1996 Mar; 7(3): | | | | |
| | | 204-21 | | | | |
| Mouse | S100b | Proc Natl Acad Sci | | | | |
| | | USA 1994 Jun 7; | | | | |
| | | 91(12): 5359-63 | | | | |
| Mouse | ATM | Genes Dev 2001 Mar | | | | |
| | | 1; 15(5): 554-66 | | | | |
| Mouse | SOD1 | Brain Pathol 1999 | | | | |
| | | Jan; 9(1): 165-86 | | | | |
| Mouse | HGPRT | J Neurochem 1999 | | Lesch-Nyhan | | |
| | | Mar; 72(3): 1139-45 | | syndrome | | |
| Mouse | HGPRT | J Mol Neurosci 2000 | | Lesch-Nyhan | | |
| | | Feb-Apr; 14(1-2): | | syndrome | | |
| | | 87-91 | | | | |
| Sheep | CLN6 | Europ J Paediatr; 5 | | | | |
| | | Neurol 2001 Suppl | | | | |
| | | A: 135-42 | | | | |
| Mouse | Dab1 | Genes Dev 1999 Mar | | | | |
| | | 15; 13(6): 643-8 | | | | |
| Mouse | reln | Genes Dev 1999 Mar | | | | |
| | | 15; 13(6): 643-8 | | | | |
| Mouse | HD94 | J Neurosci 2001 Nov | | Huntington's Disease | | |
| | | 15; 21(22): 8772-81 | | | | |
| Mouse | ApoE | Neuroscience | | | | |
| | | 1999; 94(1): 315-21 | | | | |
| Mouse | APP | Exp Neurol 2001 | | | | human APP695 |
| | | Jul; 170(1): 186-94 | | | | |
| AAV | SOD1 | J Neurochem 2002 | | Parkinson's disease | | |
| | | Jul; 82(1): 101-9 | | (PD). | | |
| Mouse | SOD1 | Neuroreport 2001 | | Alzheimer's | Primary | |
| | | May 8; 12(6): 1239-43 | | | | |
| Mouse | Ts16 | Ann N Y Acad Sci | | Down's Syndrome & | | |
| | | 1996 Jan 17; 777: | | Alzheimer's | | |
| | | 415-20 | | | | |

TABLE 3
Relevant Genes in Patents

| Gene Category | Relating to | Patent # |
|---|---|---|
| melatonin 1a and 1b receptors | Circadian rythum | 6,326,526 |
| endothelial nitric oxide synthase (eNOS) | hypertension | 6,310,270 |
| gene | | |
| alpha.4 subunit of the nicotinic | antinociceptive, hypothermia, and locomotor | 6,252,132 |
| acetylcholine receptor (nAChR) gene | effects of nicotine | |
| PS-1 and PS-2, mutations | Alzheimer's Disease | 6,395,960 |
| p19INK4d and p27KIP1 | bradykinesia, proprioceptive abnormalties, | 6,245,965 |
| | seizure-like activity | |
| TIAR gene | infertility, obesity, neurological disorders, | 6,180,849 |
| | and ovarian sex cord stromal tumors | |
| TGF-.beta.1 & (APP) gene | Alzheimer's Disease | 6,175,057 |
| Nkx-2.2 gene function and for Nkx-6.1 | decreased number of insulin and serotonin- | 6,127,598 |
| gene function | producing cells | |
| apolipoprotein E gene | apolipoprotein E-mediated pathologies | 6,046,381 |
| heterologous cholinesterase (ChE) | Alzheimer's | 6,025,183 |
| enzyme | | |
| PS1 M146L mutation & APP770 | Alzheimer's Disease (AD) | 5,898,094 |
| Huntington's Disease gene | Huntington's Disease | 5,849,995 |
| neurotrophin-3 (NT-3) | Parkinson's syndrome and Alzheimer's disease, | 5,859,311 |
| bradykinin B2 receptor gene | neurotransmitter release | 5,750,826 |
| functional DARPP-32 | schizophrenia, Parkinson's disease, and the | 5,777,195 |
| | treatment of addictions | |

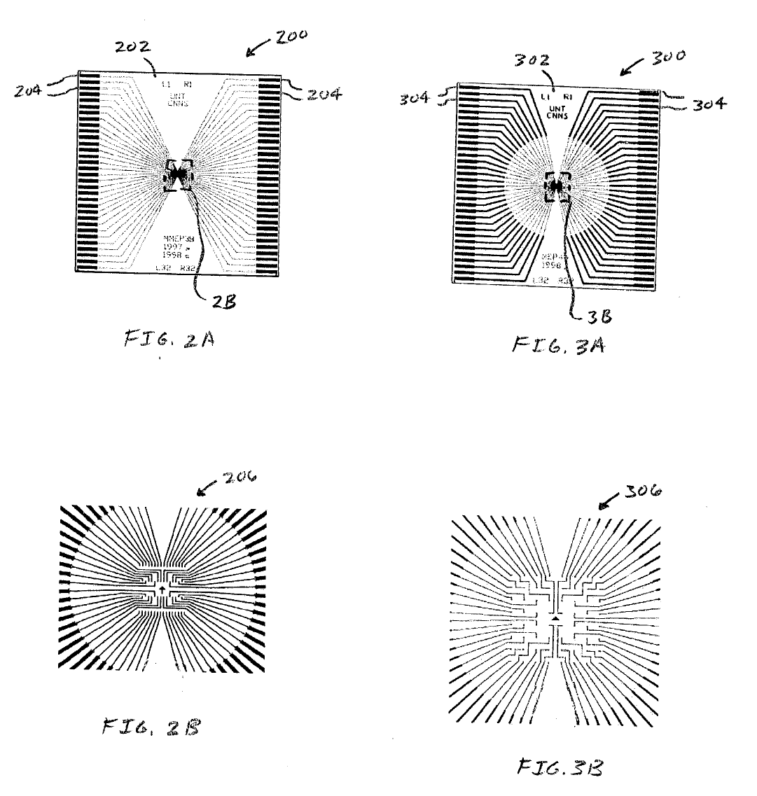

Referring now to FIGS. 2A, 2B, 3A and 3B, typical microelectrode arrays (MEA detectors) 200 and 300 that can be used in connection with the present invention are illustrated. Microelectrode array 200 is a substrate or carrier plate 202 having a number of electrodes within a recording area 206 (FIG. 2B) at the center of the substrate 202. Each electrode is electrically connected to a terminal 204 at the edge of the substrate 202. During use, the terminals are communicably coupled to the data capture unit 106 (FIG. 1). As more clearly shown in FIG. 2B, a 64 conductor MMEP 3B (product of the Center for Network Neuroscience) terminates in a 0.8 mm² recording area 206 having 4 rows of 16 columns. The electrode spacing is 40 μm between electrodes and 200 μm between rows. The electrode area is roughly 200 μm2. The carrier plate 202 measures 5×5 cm and is 1.1 mm thick.

Similarly, microelectrode array 300 is a substrate or carrier plate 302 having a number of electrodes within a recording area 306 (FIG. 3B) at the center of the substrate 302. Each electrode is electrically connected to a terminal 304 at the edge of the substrate 302. During use, the terminals are communicably coupled to the data capture unit 106 (FIG. 1). As more clearly shown in FIG. 3B, a 64 conductor MMEP 4A terminates in a 1.2 mm² recording area 306 having a matrix of 8 rows by 8 columns. Electrode spacing is equidistant at 150 μm. Electrode area is roughly 900 μm2. The carrier plate 302 measures 5×5 cm and is 1.1 mm thick.

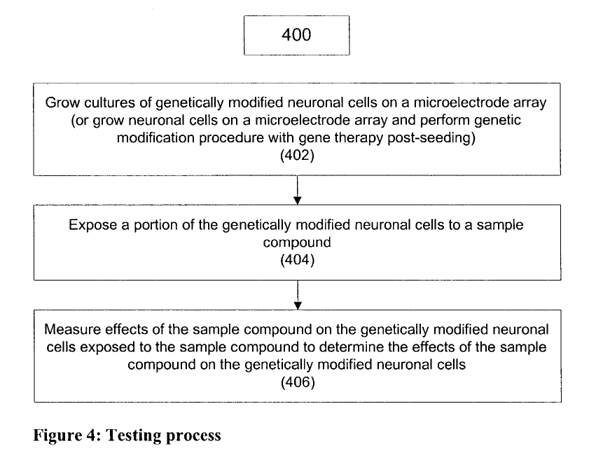

Now referring to FIG. 4, a flow chart illustrating a method 400 of determining the effects of a sample on genetically modified neuronal cells in accordance with the present invention is shown. A culture of genetically modified neuronal cells is grown on a microelectrode array in block 402. A portion of the genetically modified neuronal cells is then exposed to a sample in block 404. The effects of the sample on the genetically modified neuronal cells exposed to the sample are measured to determine the effects of the sample on the genetically modified neuronal cells in block 406.

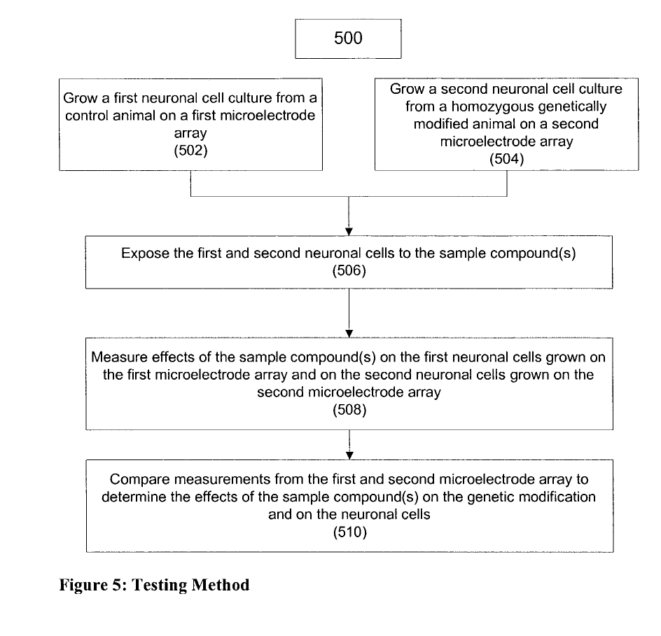

Referring now to FIG. 5, a flow chart illustrating a method 500 of determining the effects of a sample on a neuronal cell in accordance with the present invention is shown. A first neuronal cell from a control animal in grown on a first microelectrode in block 502. A second neuronal cell from a homozygous genetically modified animal is grown on a second microelectrode in block 504. The first and second neuronal cells are then exposed to a compound in block 506. The effects of the compound on the first neuronal cell with the first microelectrode and the second neuronal cell with the second microelectrode are measured in block 508. The measurements from the first and the second microelectrode are compared to determine the effects of the compound on the neuronal cell in block 510.

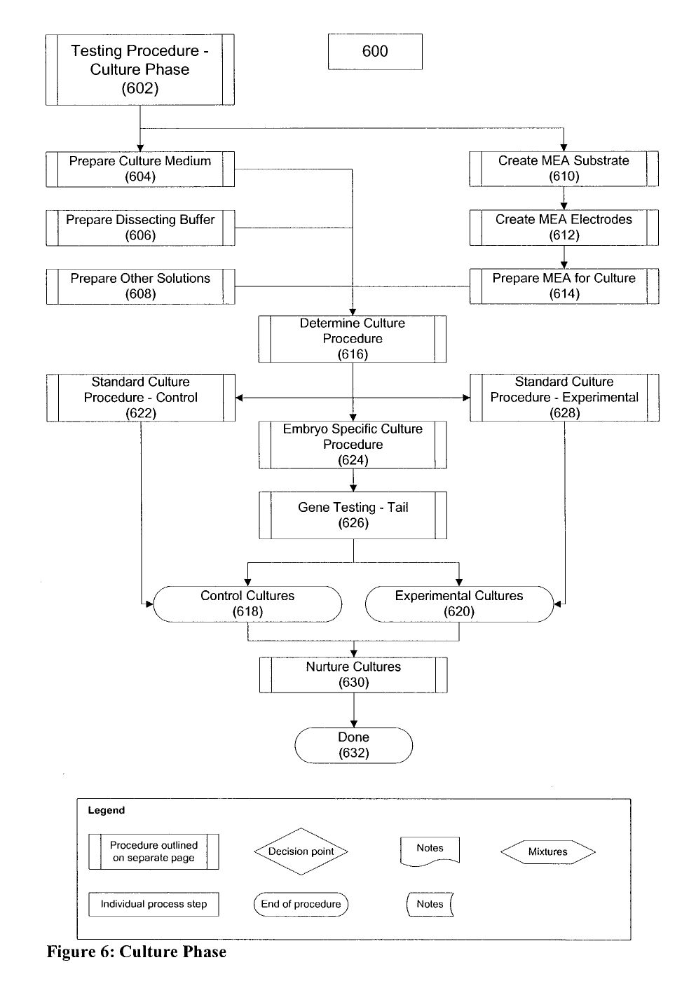

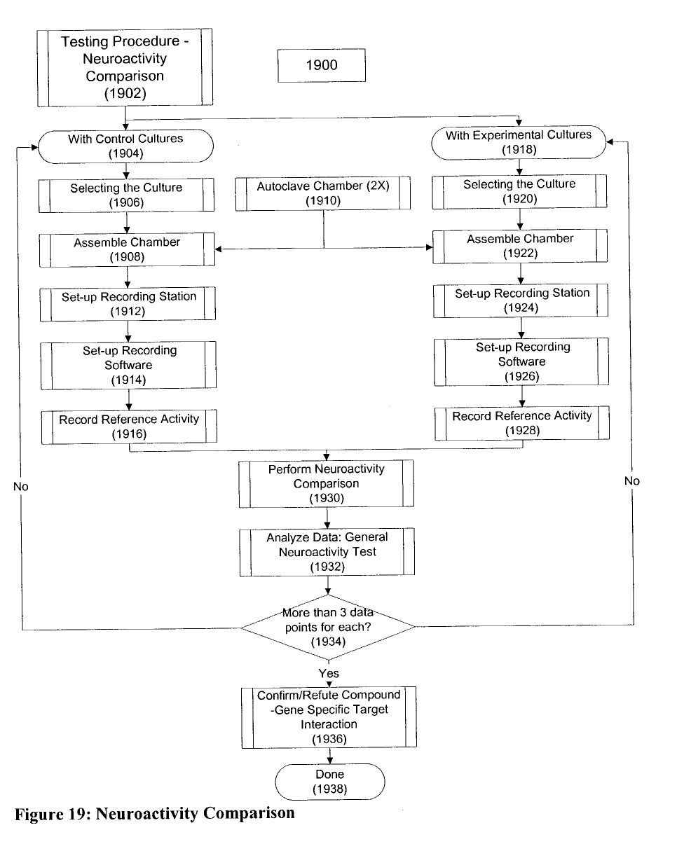

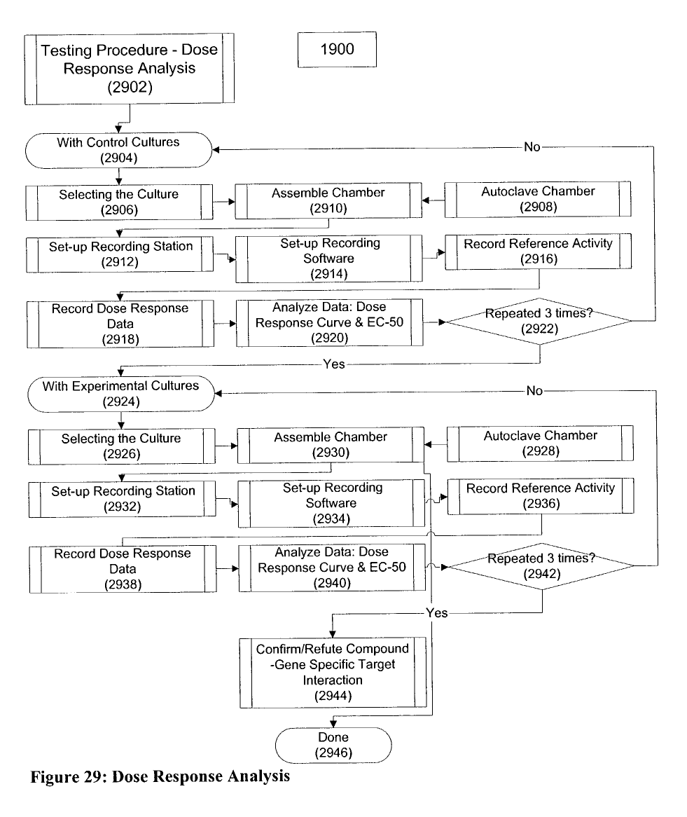

Testing procedures in accordance with various embodiments of the present invention will now be described. Specifically, testing procedures for culture phase (FIG. 6), neuroactivity comparison (FIG. 19) and response analysis (FIG. 29) are described. The microelectrode arrays are fabricated from a base of quartz coated borosilicate glass sputter coated with a layer of indium tin oxide. The indium tin oxide is photo-etched into a micro-circuit pattern and insulated with polysiloxane. The electrode pads are de-insulated with a laser and electroplated with gold. The insultation, which is normally hydrophobic, is rendered hydrophilic by flaming with a butane torch and then coated with poly-D-lysine and laminin to promote cell adhesion. Timed pregnant female mice, either transgenic or wild-type, are selected and the embryos removed. The dissection and seeding procedure is determined by the type of transgenic modification, if any. Once the target tissue has been removed from the embryos and dissociated into single cells, the cells are applied to the prepared electrode surface and maintained for at least three weeks before the cultures are used in the testing procedure. Other testing procedures in accordance with the present invention can be implemented.

Now referring to FIG. 6, a flow chart of a testing procedure for culture phase 600 is shown. Cultures for experimental testing would be prepared by fabricating the microelectrode arrays, preparing the growth surface, preparing the cells according to the required culture procedure, and maintaining the cultures until they are ready for testing.

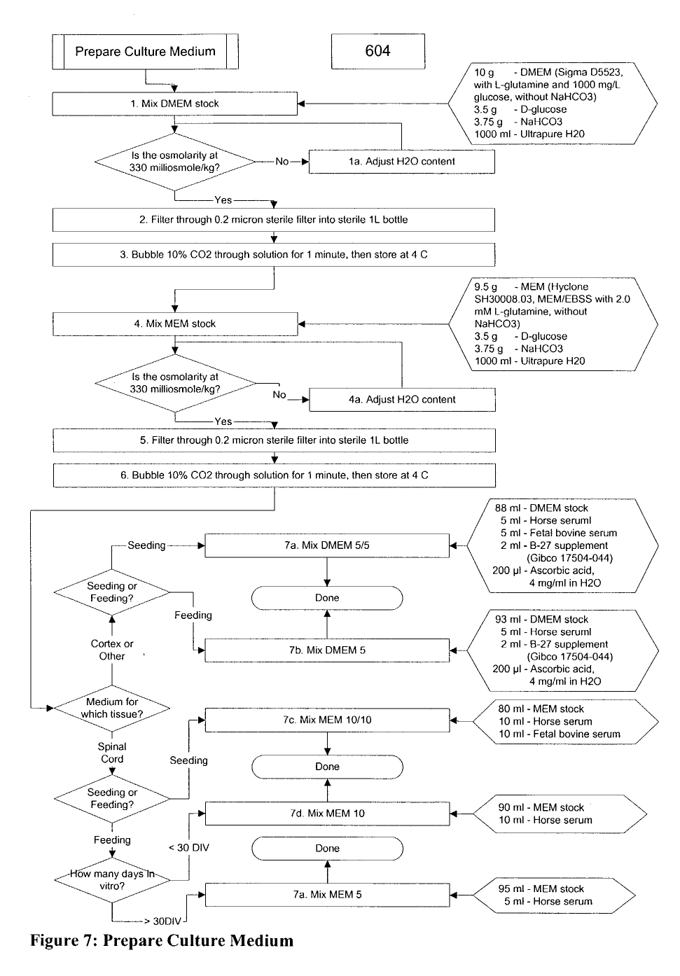

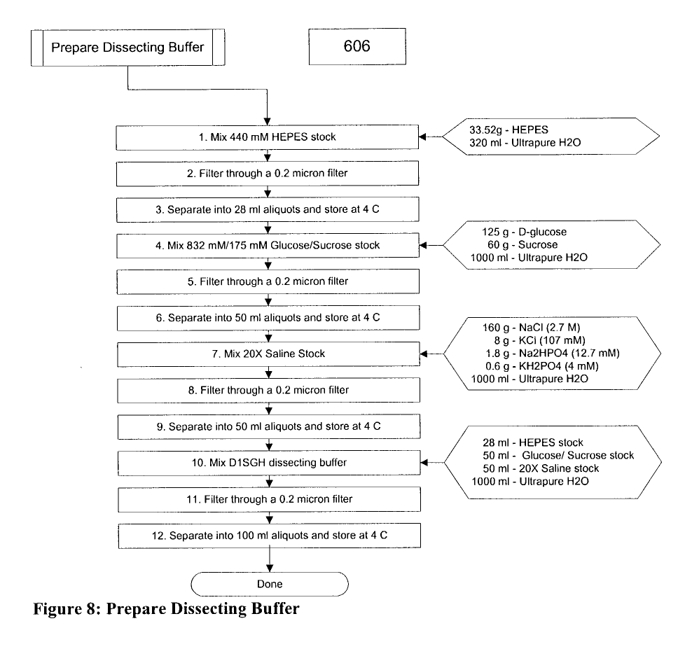

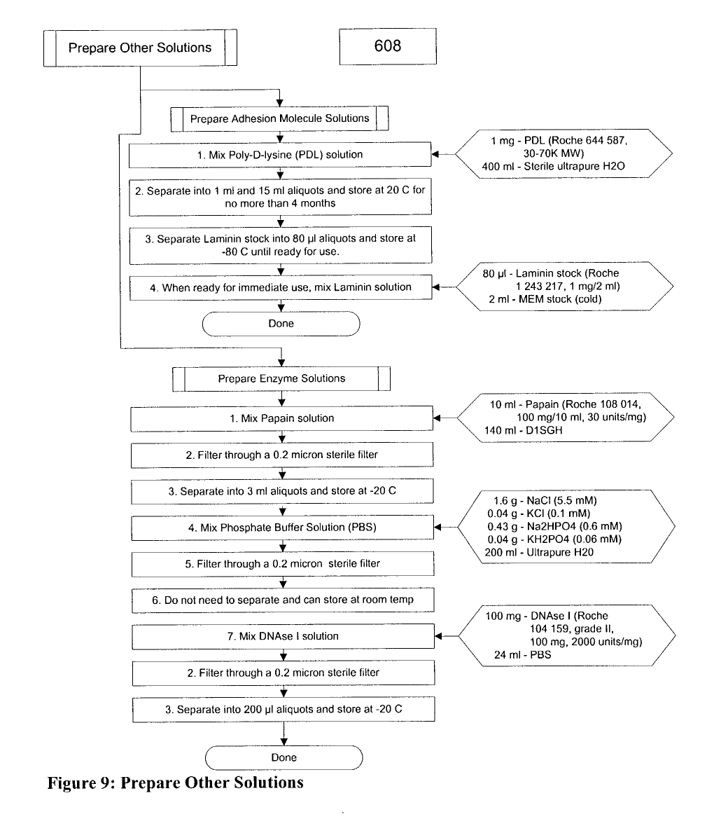

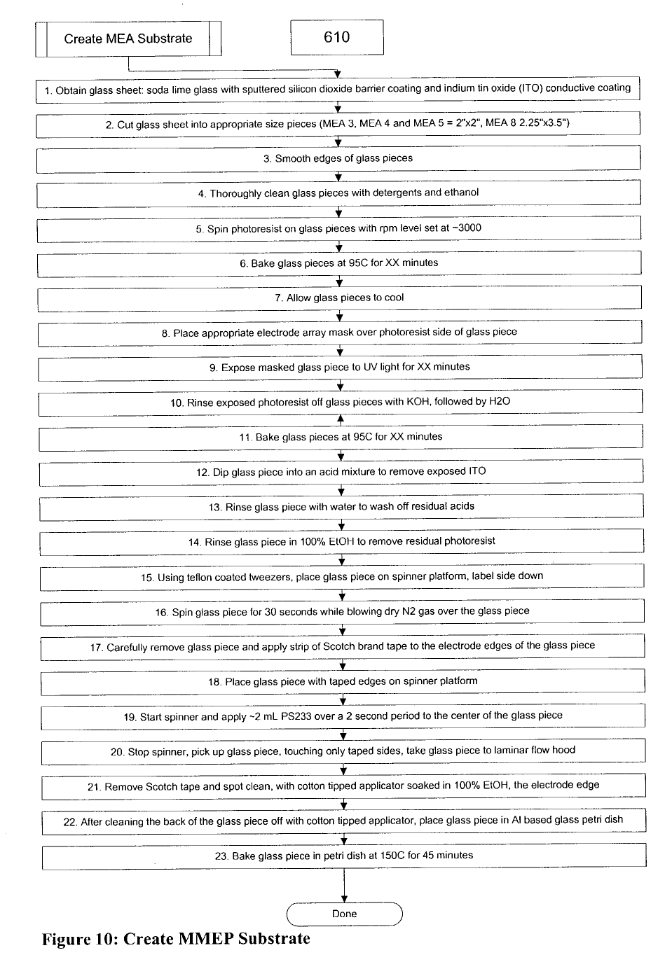

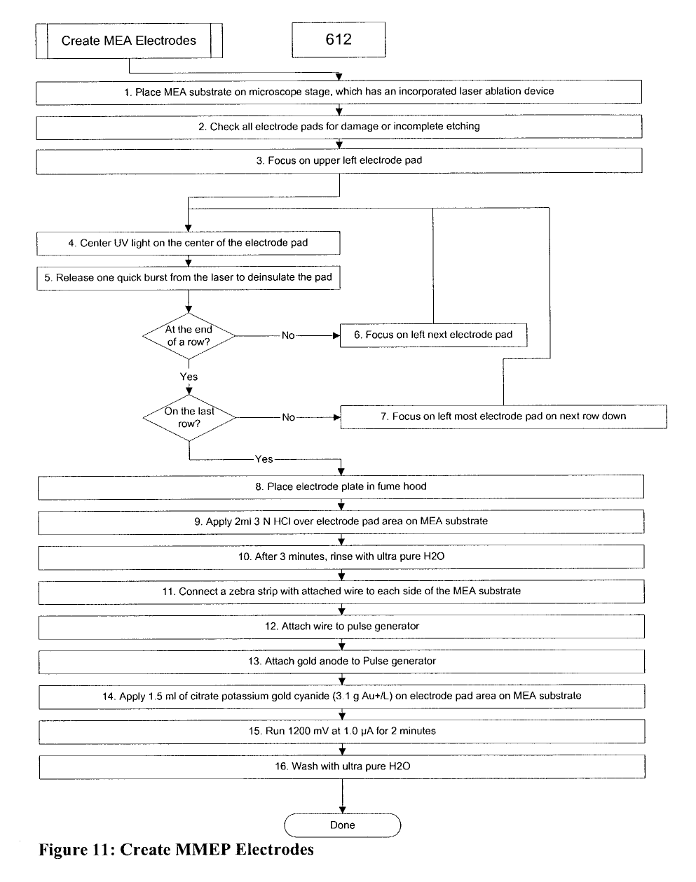

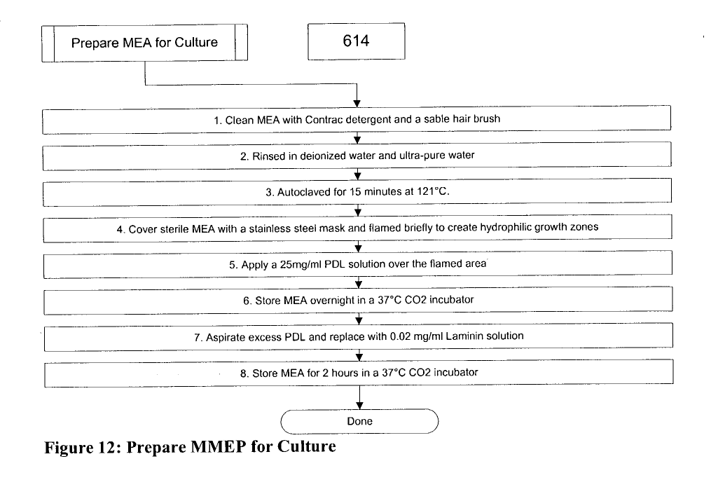

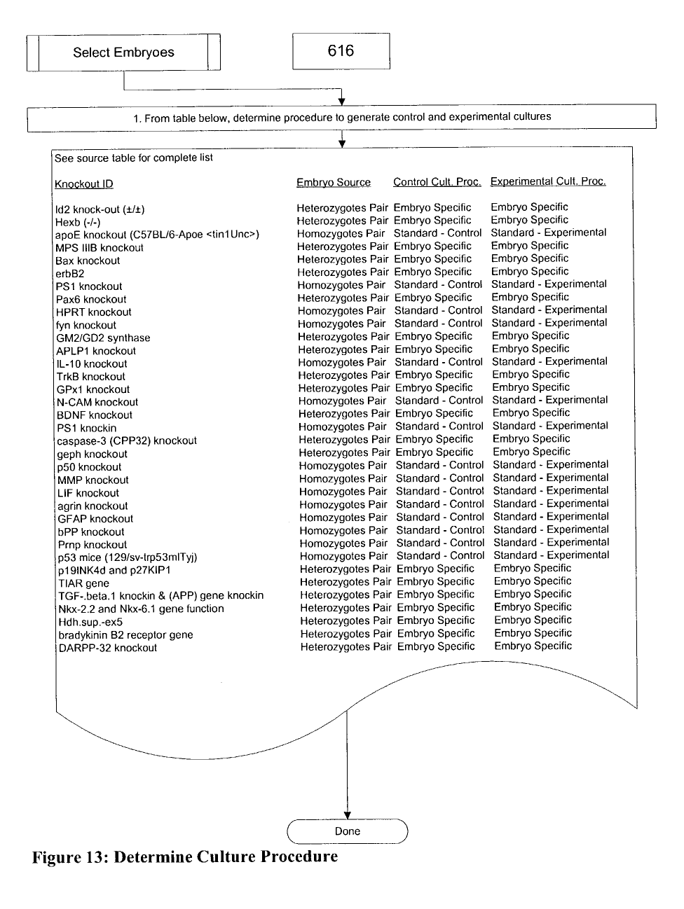

More specifically, the testing procedure for the culture phase begins in block 602. Thereafter, a culture medium is prepared in block 604 (See FIG. 7 and the corresponding description for details), the dissecting buffer is prepared in block 606 (See FIG. 8 and the corresponding description for details) and other solutions are prepared in block 608 (See FIG. 9 and the corresponding description for details). In addition, a MMEP substrate is created in block 610 (See FIG. 10 and the corresponding description for details), the MMEP Electrodes are created in block 612 (See FIG. 11 and the corresponding description for details) and the MMEP is prepared for the culture in block 614 (See FIG. 12 and the corresponding description for details). After the culture medium is prepared in block 604, the dissecting buffer is prepared in block 606, other solutions are prepared in block 608 and the MMEP is prepared for the culture in block 614, the culture procedure is determined in block 616 (See FIG. 13 and the corresponding description for details).

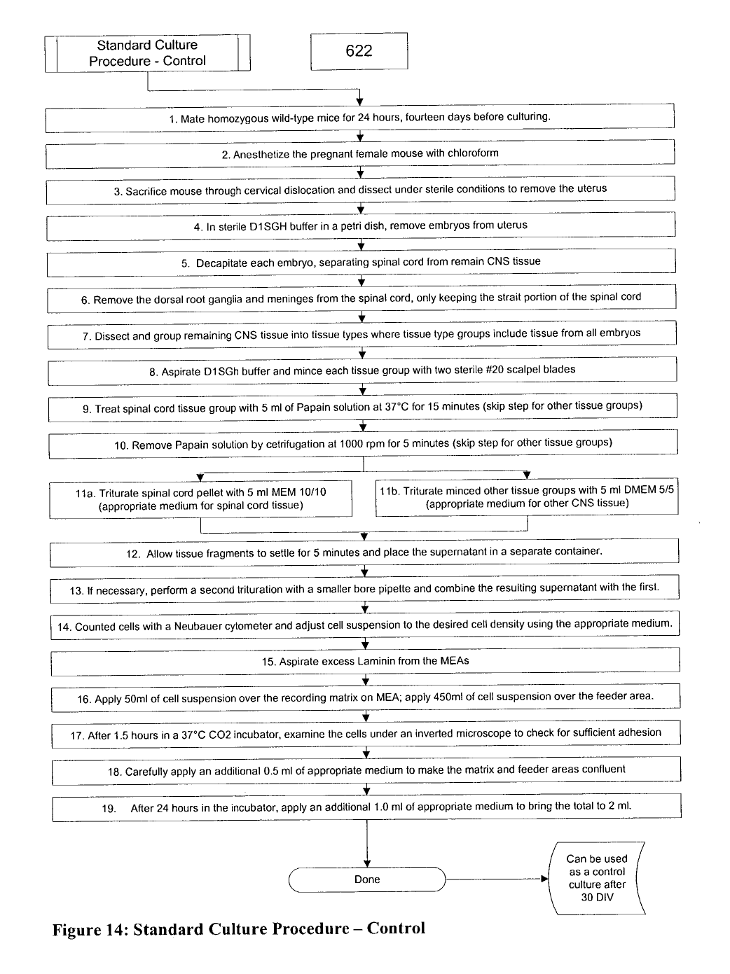

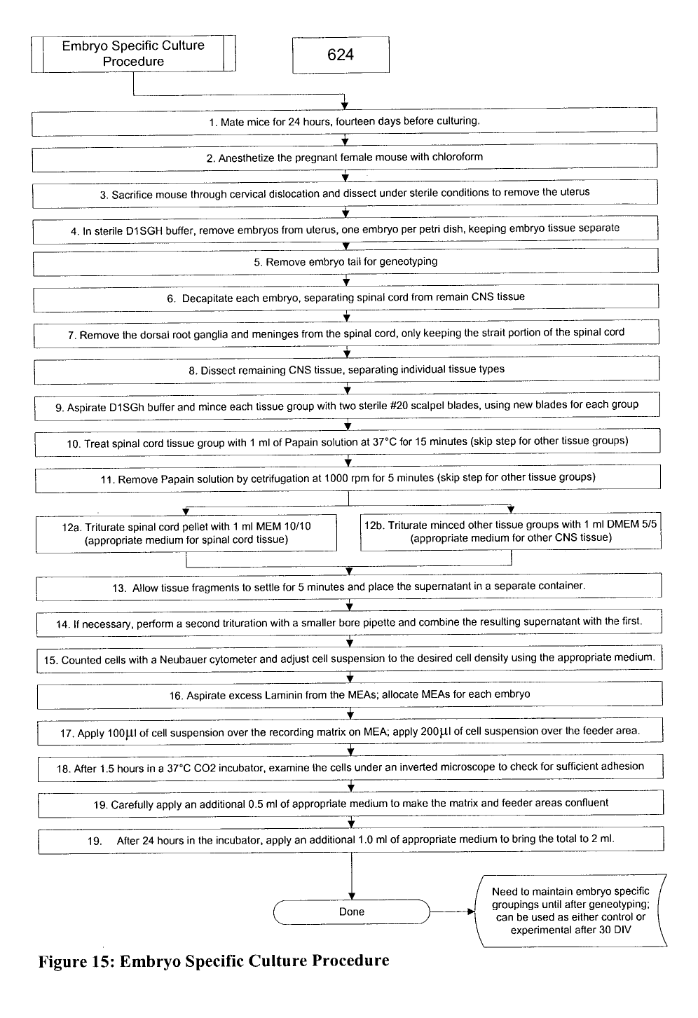

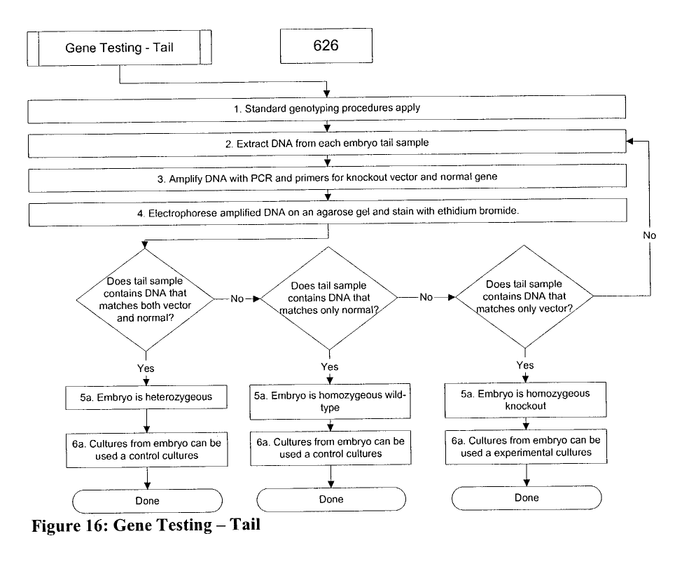

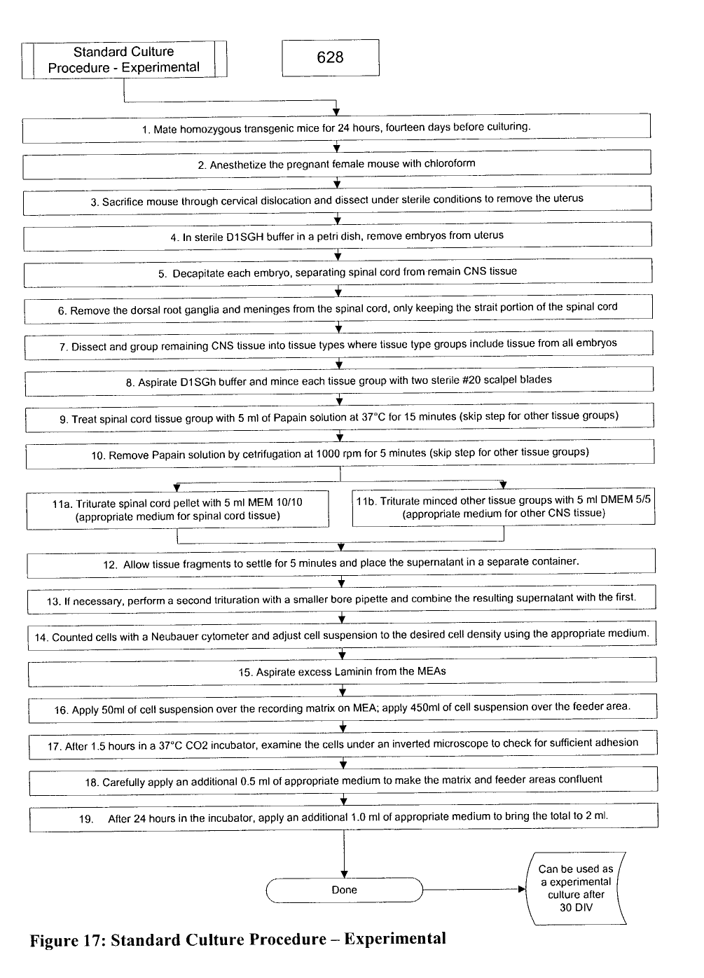

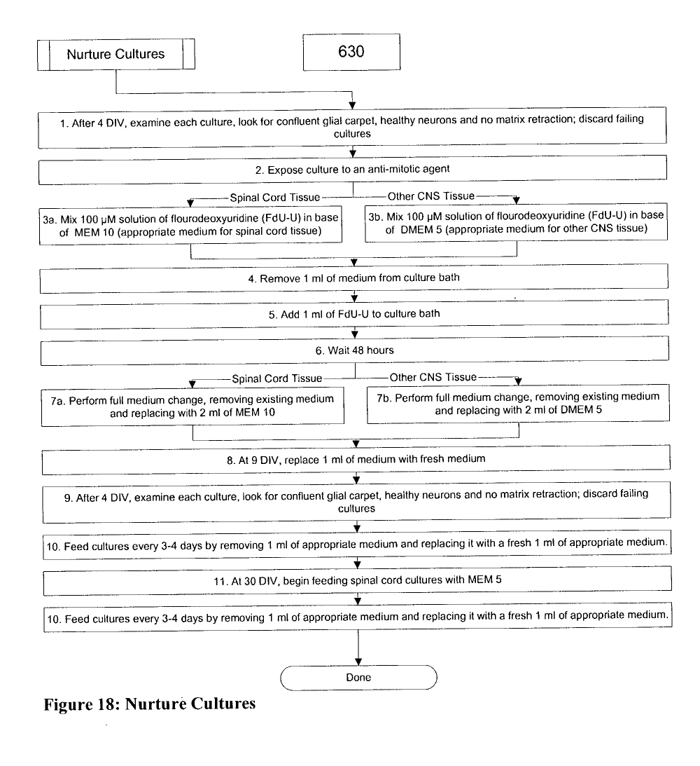

The culture procedure determination 616 identifies the method of obtaining control cultures 618 and experimental cultures 620 depending on various criteria. Control cultures 618 are created using the standard control culture procedure in block 622 (See FIG. 14 and the corresponding description for details) or an embryo specific culture procedure 624 (See FIG. 15 and the corresponding description for details) followed by gene testing (tail) in block 626 (See FIG. 16 and the corresponding description for details). Experimental cultures 620 are created using the standard experimental culture procedure in block 628 (See FIG. 17 and the corresponding description for details) or an embryo specific culture procedure 624 followed by gene testing (tail) in block 626. After the control cultures 618 and experimental cultures 620 are created, the cultures are nurtured in block 630 (See FIG. 18 and the corresponding description for details) and the process is complete in block 632.

Referring now to FIG. 7, the procedure for preparing a culture medium 604 (FIG. 6) is shown. Cell culture growth medium is prepared according to the tissue type and stage of maturity of a particular culture. Dulbecco's modified Eagle's medium (DMEM) is prepared for use with frontal cortex and auditory cortex cultures. Cortical cultures are seeded in a mixture of DMEM, 5% horse serum, and 5% fetal bovine serum. After 5 days in vitro (DIV), the fetal bovine serum is removed and the cultures are fed with DMEM and 5% horse serum only. Minimum essential medium (MEM) is prepared for use with spinal cord and hippocampal cultures. These cultures are seeded in a mixture of MEM, 10% horse serum, and 10% fetal bovine serum. After 5 DIV, the fetal bovine serum is removed and the cultures are fed with MEM and 10% horse serum only. After 30 DIV, the horse serum is cut to 5%. Both types of growth medium contain 46 mM sodium bicarbonate as a pH buffer to maintain a pH of 7.4 in equilibrium with an atmosphere containing 10% carbon dioxide.

Now referring to FIG. 8, a flow chart describing the procedure for preparing the dissecting buffer 606 (FIG. 6) is shown. A special buffer solution is prepared to maintain the embryos and tissue during the dissection procedure. The D1SGH dissecting buffer contains HEPES, to maintain a pH of 7.4 in ambient carbon dioxide, glucose to provide metabolic energy to the cells of the embryos once they have been removed from the female, and sucrose and salts to maintain the osmolarity and ionic balance of the cells. This buffer is sterilized and maintained at 4° C.

Referring now to FIG. 9, a flow chart describing the procedure for preparing other solutions (cell adhesion and enzyme solutions) 608 (FIG. 6) is shown. Other solutions are prepared for use in various stages of the procedure. The poly-D-lysine is reconstituted in sterile ultra-pure water and stored at −20° C. and thawed before it is applied to the MMEPs. Laminin is stored at −80° C. in 80 μl aliquots. It is reconstituted in 2 ml cold MEM before it is applied to the MMEPs. The papain solution is a proteolytic enzyme that is reconstituted in D1SGH and stored at −20° C. It is thawed and used in the spinal cord dissociation procedure to facilitate separation of the tissue into single cells. The DNAse solution is reconstituted in physiologic buffered saline and stored at −20° C. It is thawed and used in the dissociation procedure to lyse DNA and histone proteins released from broken cells. These molecules would otherwise cause clumping of the cells and prevent an even monolayer from forming.

Now referring to FIG. 10, a flow chart describing the procedure for creating the microelectrode array (MEA) substrate 610 (FIG. 6) is shown. The microelectrode arrays (MEAs or MMEPs) are created through a standard lithography process. The MMEP is cut from indium tin oxide (ITO) coated soda lime glass. 2 inch by 2 inch pieces of glass are cut and the edges are smoothed. After a thorough cleaning, photo resist is spun on the glass piece and the glass is baked. After cooling, the MMEP mask is placed over the photo resist covered glass and the glass is exposed to UV light. Exposed photo resist is then washed from the glass with KOH and the glass is rinsed with water. The patterned glass is dipped in an acidic solution to remove the exposed ITO. The remaining photo resist is removed with 100% EtOH and the ITO patterned glass is prepared for deposition of the poly-siloxane (PS233) coating by covering the zebra stiped edges with tape. PS233 is spun on the patterned glass and baked to harden the PS233 insulation layer.

Referring now to FIG. 11, a flow chart describing the procedure for creating electrodes on the MEA substrate 612 (FIG. 6) is shown. Once the ITO glass is patterned and coated with insulation, it is ready for the electrode process. To create the electrodes, the ITO electrode pads under the insulation layer are exposed with laser ablation. A laser is focused on each electrode pad and fired for a short burst to ablate the insulation layer from the electrode. Once each electrode pad on the MMEP is uncovered, the MMEP is dipped in citrate potassium gold cyanide and the exposed electrode pads are electroplated. A pulse generator is connected to the zebra stripes at the edge of the MMEP to provide the current. Once electroplated, the MMEP is cleaned and is ready for use.

Now referring to FIG. 12, a flow chart describing the procedure for preparing the MEA for cell cultures 614 (FIG. 6) is shown. The MMEP insulation substrate must be prepared to allow the growth of the neuronal network. The surface of the MMEP must be cleaned with a gentle detergent to remove any residue that might inhibit the growth of the cultures, while preserving the integrity of the insulation and maintaining optical clarity. The MMEPs are sterilized by autoclaving and flamed with a butane torch to generate a hydrophilic growth surface. Poly-D-lysine and laminin are applied to promote cell adhesion.

Referring now to FIG. 13, a flow chart describing the procedure for determining the cell cultures to be created (selecting embryos) 616 (FIG. 6) is shown. The type of transgenic modification selected will determine which culture procedure must be used to obtain the appropriate cultures. When a particular transgenic modification is selected to model a given disease state, the source of the embryos will determine the culture procedure that must be used. If we use wild type animals to supply control cultures, we will use the standard culture procedure. If we use embryos derived from a homozygous transgenic mating pair, we will also use the standard culture procedure. If we use embryos derived from a heterozygous transgenic mating pair, we will keep the tissue from individual embryos separate so that we can correlate the cultures derived from each embryo with the genotype results from each embryo.

Now referring to FIG. 14, a flow chart describing the procedure for preparing a cell culture to be used for the standard control culture 622 (FIG. 6) is shown. In the standard culture procedure for control cultures, tissue from all embryos is pooled to produce a common cell suspension, which is then seeded on prepared MMEPs. Timed pregnant female mice are anesthetized and the embryos are removed. The target tissue is dissected from each embryo and pooled. Spinal cord is treated with a proteolytic enzyme for 15 minutes and then mechanically disrupted into a single cell suspension. Other tissues are mechanically disrupted without enzymatic treatment. The cell suspension is seeded onto the prepared MMEPs and allowed to settle for one hour. After one hour, the cultures are filled with 2 ml of medium.

Referring now to FIG. 15, a flow chart describing the procedure for preparing an embryo specific culture 624 (FIG. 6) is shown. The standard culture procedure for experimental cultures follows the same protocol as for control cultures, described in FIG. 14.

Now referring to FIG. 16, a flow chart describing the procedure for gene testing - tail 626 (FIG. 6) is shown. The embryo specific culture procedure is used if the embryos must be obtained from a heterozygous transgenic mating pair. Tissue dissection is performed as in the standard protocol, except that tissue from different embryos is not pooled. Tissue from individual embryos is dissociated in a small volume of medium and seeded onto a defined set of prepared MMEPs. Tail tissue from each embryo is collected and genotyped (homozygous transgenic, heterozygous, or homozygous wild type) for later correlation with the cultures resulting from that embryo.

Referring now to FIG. 17, a flow chart describing the procedure for preparing a standard experimental culture 628 (FIG. 6) is shown. DNA from the tail of each embryo from a heterozygous transgenic mating must be tested to determine the genotype of each embryo. Tail tissue is collected from each embryo and the DNA is extracted. The DNA is amplified by PCR using primers that recognize the transgenic modification in question. Amplified DNA is separated by agarose gel electrophoesis and the genotype of the embryo is determined by the presence of absence of specific DNA sequences.

Now referring to FIG. 18, a flow chart describing the procedure for nurturing the cultures 630 (FIG. 6) is shown. Cultures will be treated to control glial cell growth and maintained for at least one month before experimental use. After 4 days in vitro, cultures are treated with an anti-mitotic agent to prevent the proliferation of glial cells. After 6 DIV, this agent is washed out with a full medium change, and the cultures are fed three times per week subsequently by half medium changes. After one month, the cultures may be used for experiments.

Referring now to FIG. 19, a flow chart outlining the basic steps in the neuroactivity testing procedure 1900 is shown. A neuroactivity comparison test is performed to test a compound's effect on a certain gene (including any cellular properties, which may be a certain receptor, ion channel, signaling protein or other cellular property, and mechanisms that result from the presence of that gene). The compound is tested on a control culture, usually from a wild-type or heterozygous animal, as well as an experimental culture, usually from a genetically modified animal (knock-out or knock-in). The neuroactivity comparison test includes analysis of spike rate, burst rate, spike waveform shape, burst intervals, and other standard electrophysiological data, and compares the differences between the control culture and the experimental culture.

The testing process starts with the selection of a culture and the preparation of the testing chamber. Once the testing chamber is installed into the test station, reference activity is recorded to establish the baseline neuroactivity of the culture. Every culture forms slightly different network connections and therefore has different levels of spontaneous activity. However, each network is capable of responding to a pharmacological agent in a representative manner. Once a base line is recorded, the test compound is applied simultaneously to both the control and experimental culture, and changes in the cultures neuroactivity are recorded. If the compound elicits different reactions in the control and experimental culture, it signifies that the compound has differential effects on the cultures due to an interaction with the properties and/or mechanisms of the modified gene. Changes in each culture's individual base line are compared, and analyzed with in-house and commercially available software and tested for statistical significance (using the standard t-test or other appropriate statistics).

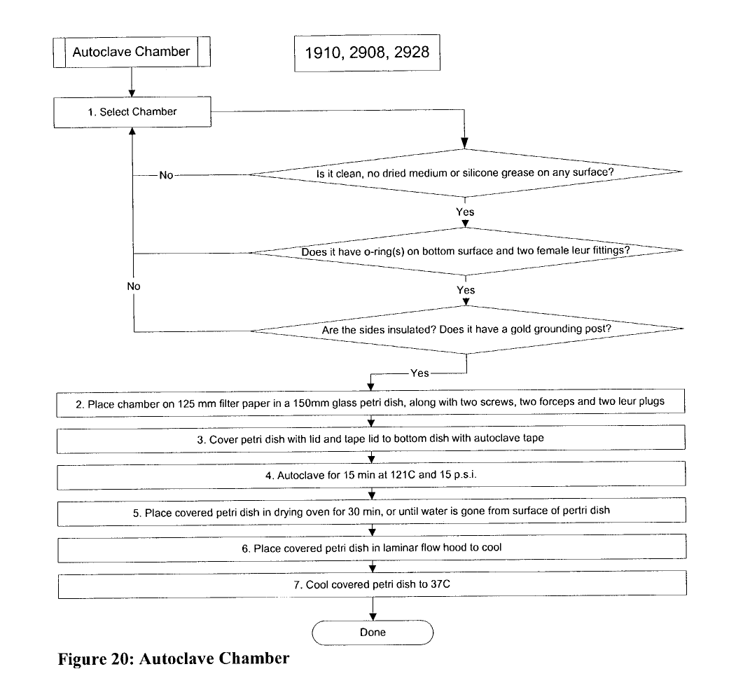

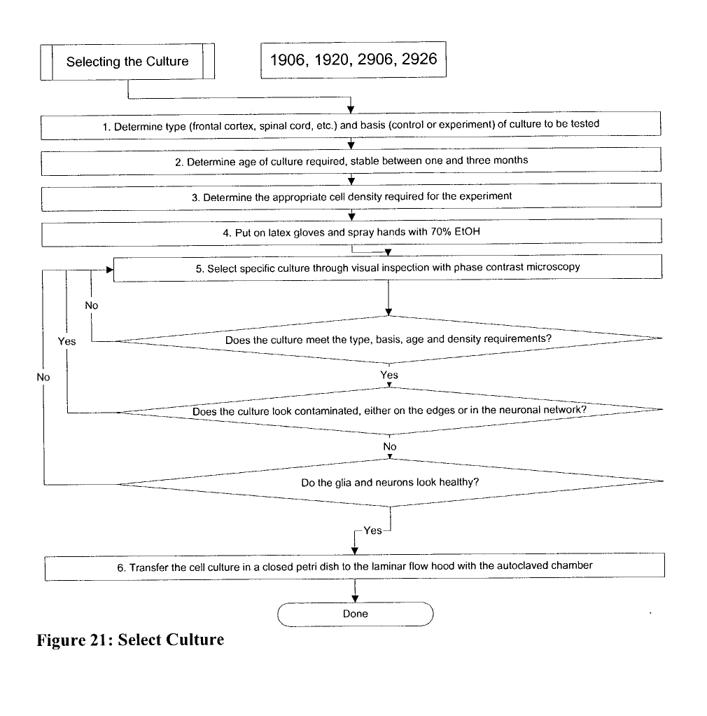

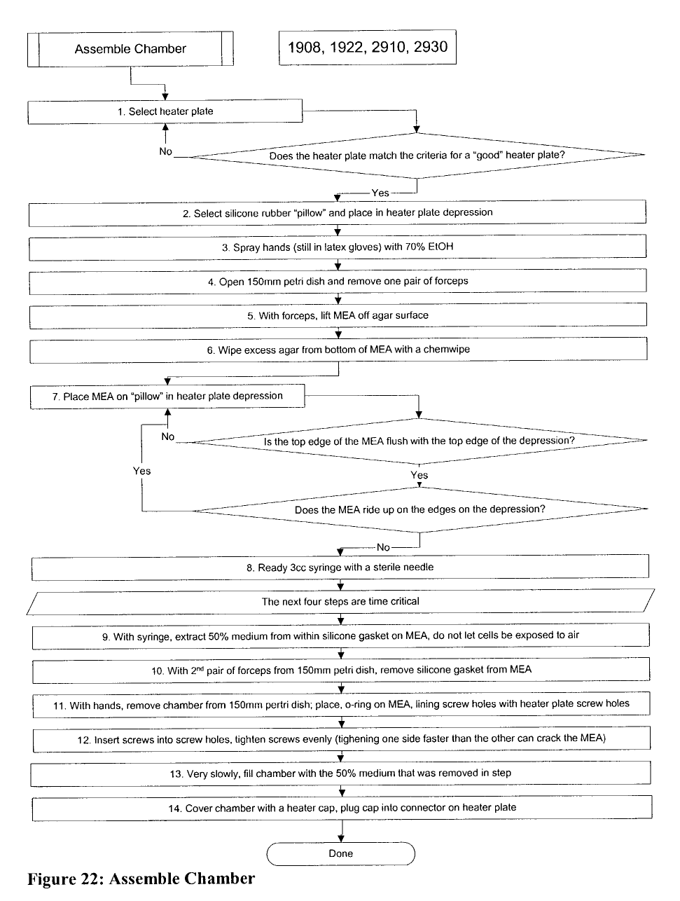

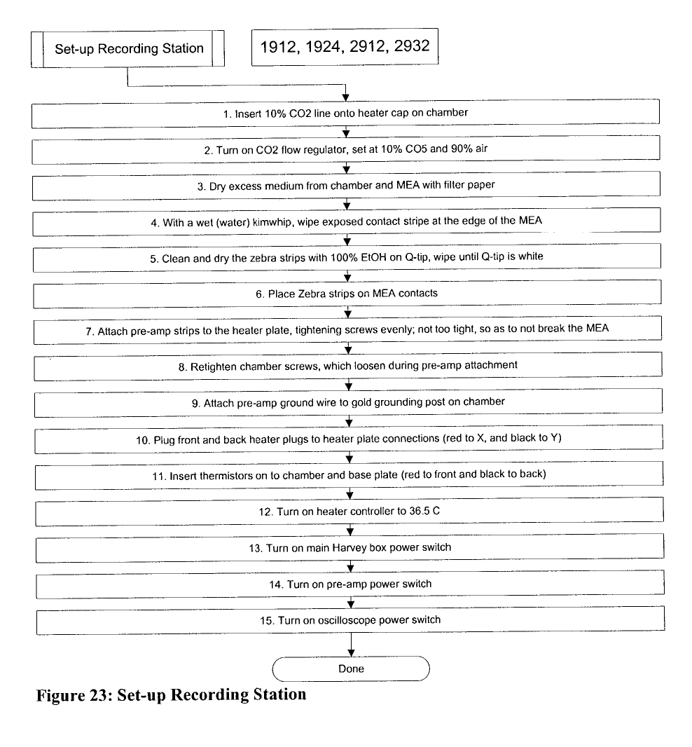

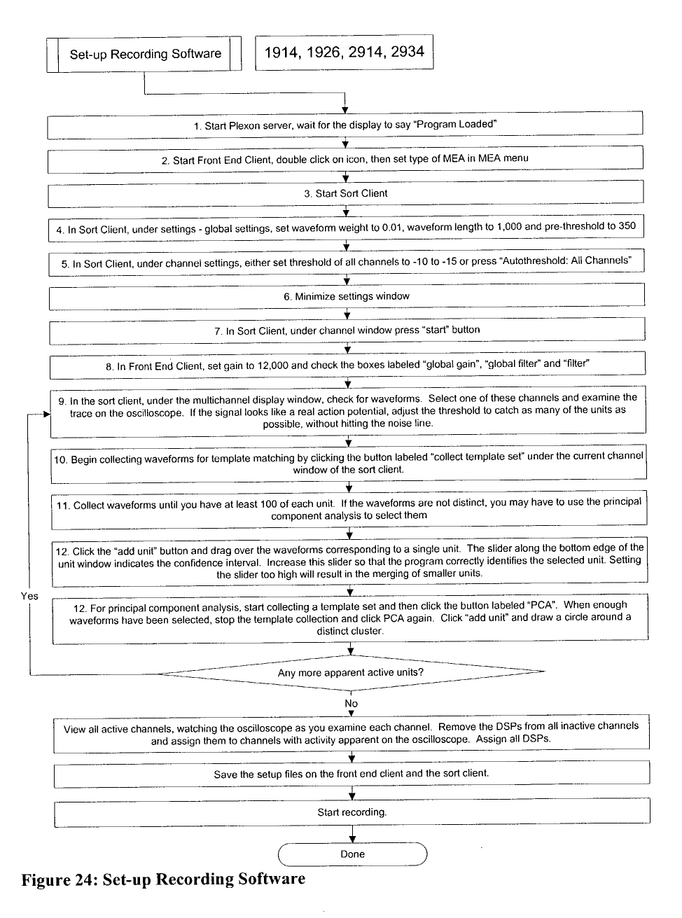

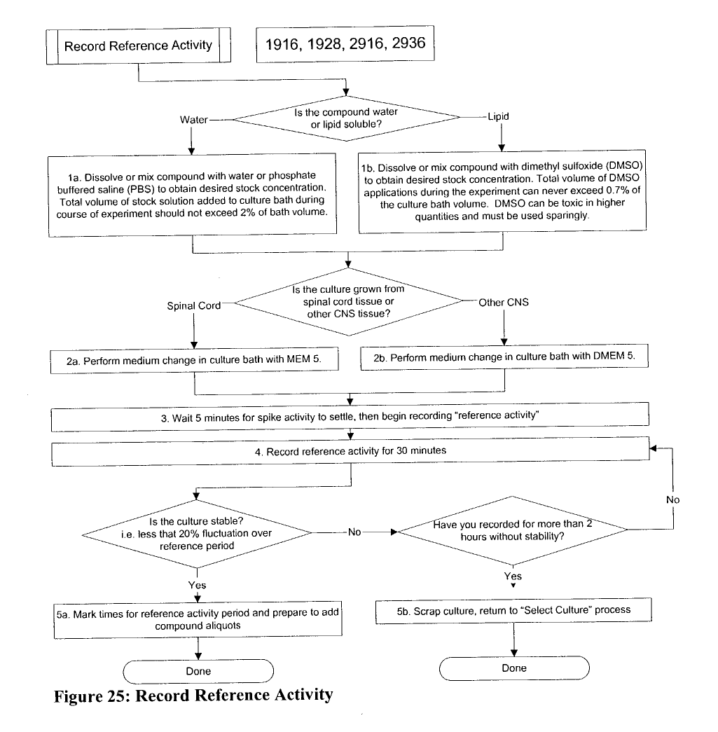

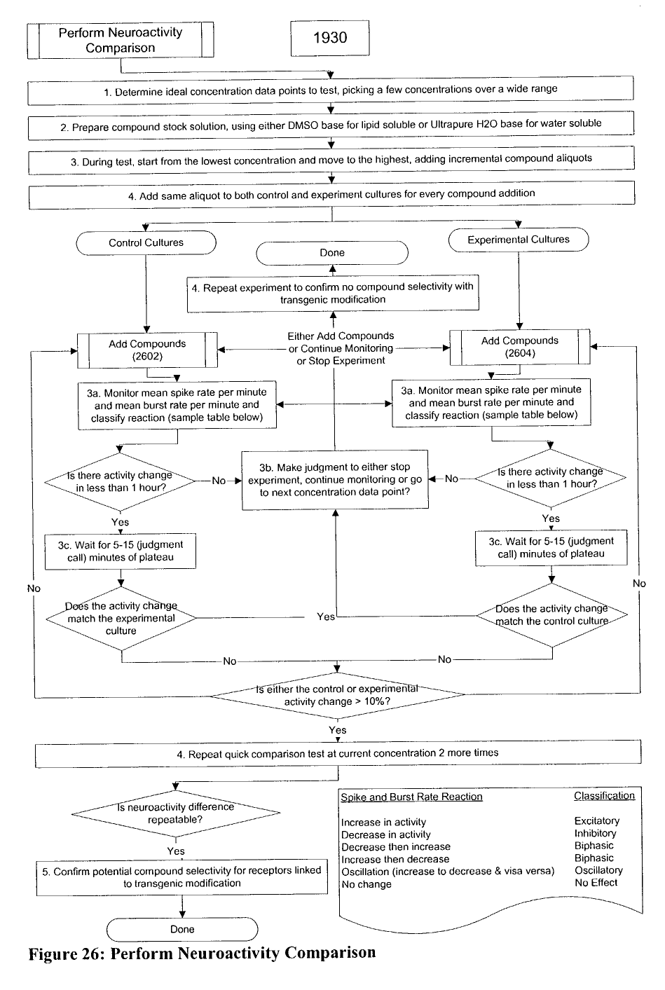

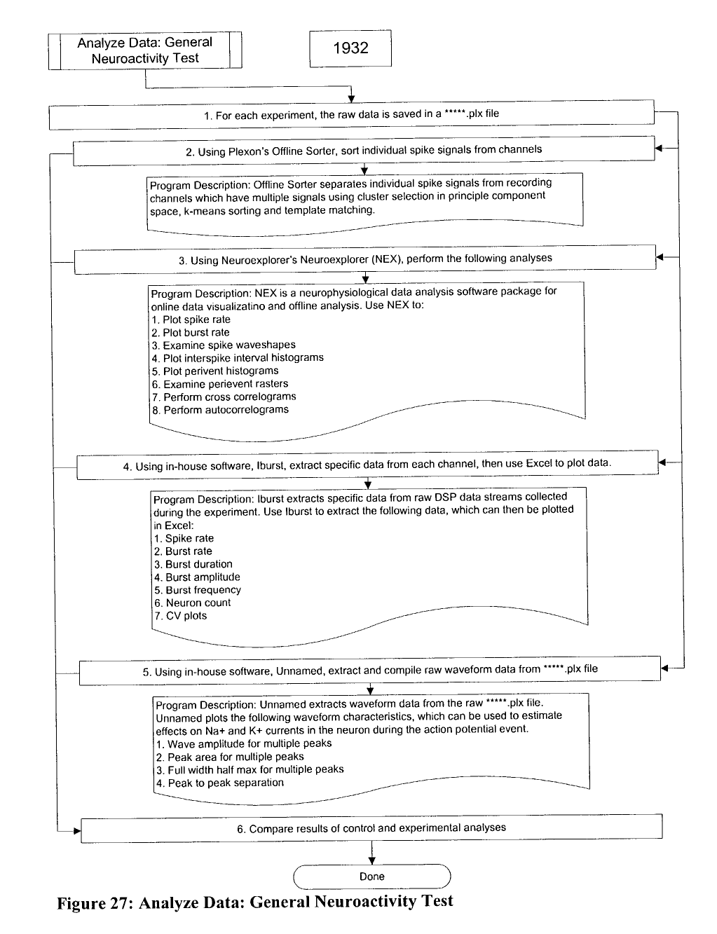

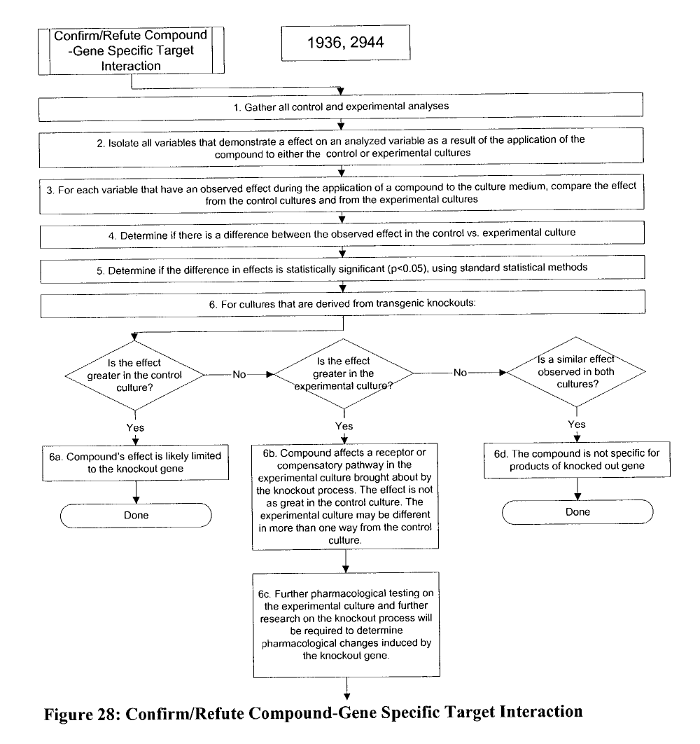

More specifically, the neuroactivity comparison testing procedure 1900 begins in block 1902 where two parallel procedures are conducted for control cultures 1904 and experimental cultures 1918. The control culture procedure 1904 begins with selecting the culture 1906 (See FIG. 21 and the corresponding description for details) and autoclaving the testing chamber 1910 (See FIG. 20 and the corresponding description for details). Once those steps are complete, the testing chamber is assembled in block 1908 (See FIG. 22 and the corresponding description for details). The recording station is setup in block 1912 (See FIG. 23 and the corresponding description for details), the recording software is setup in block 1914 (See FIG. 24 and the corresponding description for details) and the reference activity is recorded in block 1916 (See FIG. 25 and the corresponding description for details). Similarly, the experimental culture procedure 1918 begins with selecting the culture 1920 (See FIG. 21 and the corresponding description for details) and autoclaving the testing chamber 1910 (See FIG. 20 and the corresponding description for details). Once those steps are complete, the testing chamber is assembled in block 1922 (See FIG. 22 and the corresponding description for details). The recording station is setup in block 1924 (See FIG. 23 and the corresponding description for details), the recording software is setup in block 1926 (See FIG. 24 and the corresponding description for details) and the reference activity is recorded in block 1928 (See FIG. 25 and the corresponding description for details). A neuroactivity comparison is then performed on the control cultures and the experimental cultures in block 1930 (See FIG. 26 and the corresponding description for details) and the comparison data is analyzed in block 1932 (See FIG. 27 and the corresponding description for details). If more than three data points are obtained for each culture, as determined in decision block 1934, the compound-gene specific target interaction is confirmed or refuted in block 1936 (See FIG. 28 and the corresponding description for details) and the procedure ends in block 1938. If, however, the more than three data points are not obtained for each culture, as determined in decision block 1934, the control culture procedure 1904 and/or the experimental culture procedure 1918 is repeated.

Now referring to FIG. 20, a flow chart describing the procedure to autoclave the testing chamber 1910, 2908 and 2928 (FIGS. 19 and 29) is shown. Recording chambers must be selected and sterilized prior to use. An appropriate recording chamber must be selected and determined to be clean and in proper working order before it is used. The selected chamber must be sterilized by autoclaving at 121° C. for 15 minutes at 15 p.s.i. The chamber must then be dried in a 70° C. oven and allowed to cool to no more than 37° C.

Referring now to FIG. 21, a flow chart describing the procedure to select the cell culture to be used for testing 1906, 1920, 2906 and 2926 (FIGS. 19 and 29) is shown. An appropriate culture must be selected for the experiment. A culture must meet certain criteria before it may be selected for use in an experiment. After we determine the appropriate tissue type, we select a culture that is between one and three months old. We visually inspect it under a phase contrast microscope to determine if the density of cells is adequate and that the cells are healthy.

Now referring to FIG. 22, a flow chart describing the procedure to assemble the testing chamber 1908, 1922, 2910 and 2930 (FIGS. 19 and 29) is shown. Once the recording chamber has been sterilized and the culture has been selected, the recording chamber, MMEP with cell culture and base plate are assembled into one unit. After an appropriate base plate and MMEP silicone rubber pillow is selected, all pieces are placed in a laminar flow hood. Lift MMEP from petri dish and place on pillow, which is one the base plate. Very quickly, remove half of the medium from the silicone gasket that is over the cells, remove the silicone gasket, put the chamber over the gasket's previous location and add the removed medium to the opening in the chamber over the cells. Cover the open chamber with a heater cap and move the base plate and chamber to the testing station microscope stage.

Referring now to FIG. 23, a flow chart describing the procedure to set up the testing station 1912, 1924, 2912 and 2932 (FIGS. 19 and 29) is shown. Once on the testing station microscope stage, the base plate and chamber are connected to the system and prepared for testing. A 10% CO2 line is plugged into the heater cap. Excess medium is dried from the base plate with filter paper. The zebra stripes are wiped clean with EtOH before the pre-amplifiers are attached and clamped down. Grounding wires, heating wires and thermistors are plugged into the base plate and pre-amplifier. The heater controller is set to 36.5 C and the electronic components are turned on, including the Plexon system and the oscilloscope.

Now referring to FIG. 24, a flow chart describing the procedure to set up the testing software 1914, 1926, 2914 and 2934 (FIGS. 19 and 29) is shown. Once the biological components are connected to the data acquisition electronics, the software can be set up and the active channels can be identified. The Plexon data acquisition software is loaded, as well as other monitoring and analysis programs. Using the standard Plexon procedures, active electrodes are identified and DSP's are assigned to individual waveform patterns. Each electrode could have as many as four individual waveform patterns, representing different nerve cell signals. Once all of the active units are identified and the DSP's assigned, the data recording starts.

Referring now to FIG. 25, a flow chart describing the procedure to record the reference activity 1916, 1928, 2916 and 2936 (FIGS. 19 and 29) is shown. The beginning of every testing includes a recording at least 30 minutes of reference activity after a medium change. The medium, the type will depend upon the type of tissue used to create the cell culture, should be replaced in small increments to minimize any turbulence effects from the liquid movement in the chamber. After a full replacement of medium, reference activity recording begins, and ends after 30 minutes of statistically stable activity. If statistically stable activity can mot be obtained within two hours, the culture should be scrapped and a new culture should be prepared for testing.

Now referring to FIG. 26, a flow chart describing the procedure to perform the testing needed for the neuroactivity comparison 1930 (FIG. 19) is shown. A basic neuroactivity comparison is made by testing a control culture, usually from a wild-type or heterozygous animal, and an experimental culture, usually from a genetically modified animal. A series of concentrations are selected that have known effects on the neuroactivity of a control culture. Compounds are added to each culture and the neuroactivity reaction is recorded. Reactions may take up to 2 to 3 hours to occur. If there are statistically significant differences in the basic neuroactivity changes, as defined by excitatory, inhibitory, biphasic, oscillatory or no effect, induced by the compound aliquots, then the compound likely has an effect on the receptors that are linked to the genetic modification of the genetically modified animal culture. The comparison is repeated to confirm the neuroactivity difference.

Referring now to FIG. 27, a flow chart describing the procedure to analyze the neuroactivity comparison data 1932 (FIG. 19) is shown.

Now referring to FIG. 28, a flow chart describing the procedure to use the data to determine if a compound targets the products of a specific gene 1936 and 2944 (FIGS. 19 and 29) is shown.

Referring now to FIG. 29, a flow chart outlining the basic steps in the dose response analysis testing procedure 2900 is shown. A more comprehensive test for determining a compounds effect on a certain gene, including any cellular properties, which may be a certain receptor, ion channel, signaling protein or other cellular property, and mechanisms that result from the presence of that gene, is the comparison of the dose response curve for the compound on the control and experimental cultures. The compound is tested at 5-8 neuroactivity-changing concentrations on both a control culture, usually from a wild-type animal, and an experimental culture, usually from a genetically modified animal (homozygous or heterozygous for the gene of interest). The dose response curves are a plot of concentration versus percent change in base level spontaneous neuroactivity. Dose responses are usually plotted on a semi-logarithmic scale, with percent change of neuroactivity on the y-axis (usually calculated from average network spike rate and/or average network burst rate divided by the base line levels recorded during the reference activity) and the concentration of compound applied on the x-axis. Different dose response curves in the control and experimental culture signify that the compound has an interaction with the properties and/or mechanisms of the modified gene.

The testing process starts with the selection of a culture and the preparation of the testing chamber. Once the testing chamber is installed into the test station, reference activity is recorded to establish the baseline neuroactivity of the culture. Once a base line is recorded, up to 8 different concentrations of the compound is added simultaneously to both the control and the experimental culture (with approximately half hour to 1 hour between each addition), and the changes in the cultures neuroactivity are recorded. The average spike and burst rates at the different concentrations are plotted as a percent change from reference activity. For example the addition of an inhibitory compound to the culture medium results in an overall 30% reduction of spike rate from reference activity (which is considered 100%), This change from reference activity is represented as a single data point at 70% (y-axis), corresponding to the concentration applied x-axis). This process is repeated for the other 7 concentrations until 8 points can be plotted and a dose response curve can be derived from the points. An effect can be confirmed if the dose response curves of the compound for the control and experiment cultures are statistically different, as proven by standard statistical methods.

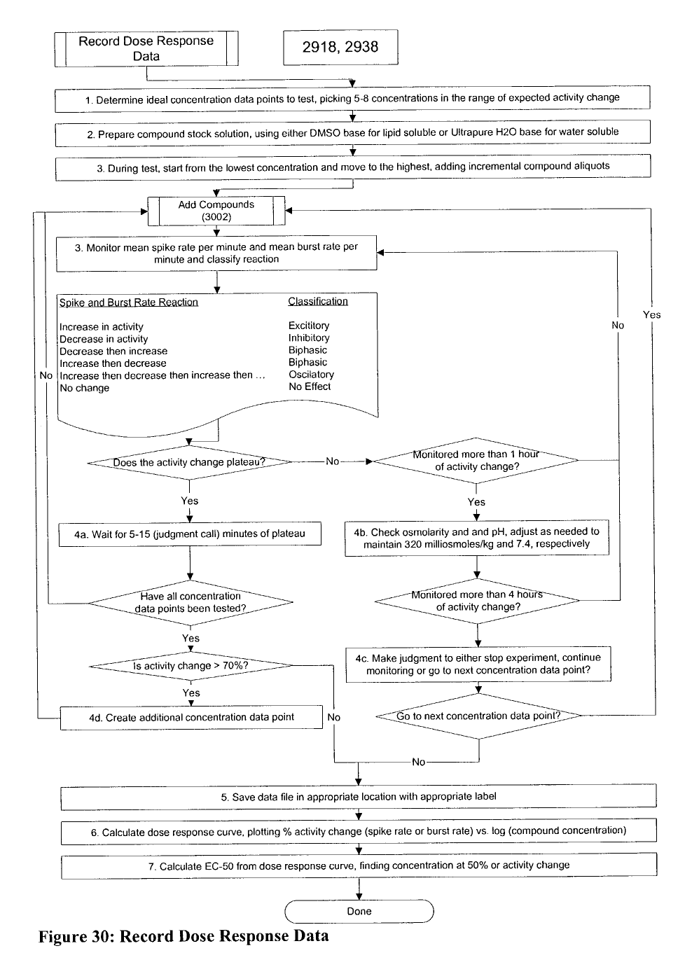

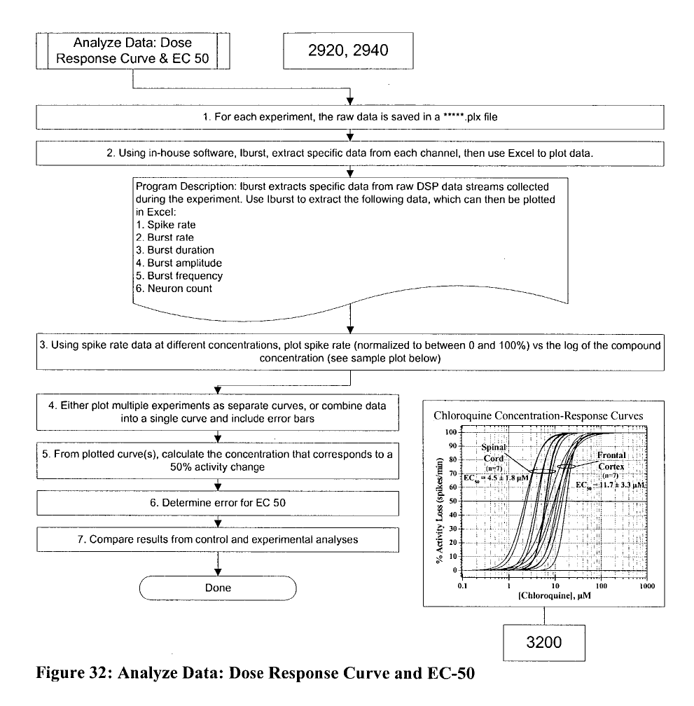

More specifically, the dose response analysis testing procedure 2900 begins in block 2902 and involves two parts—control cultures 2904 and experimental cultures 2924. For the control cultures 2904, the culture is selected in block 2906 (See FIG. 21 and the corresponding description for details), the testing chamber is autoclaved in block 2908 (See FIG. 20 and the corresponding description for details) and the testing chamber is assembled in block 2910 (See FIG. 22 and the corresponding description for details). Thereafter, the recording station is setup in block 2912 (See FIG. 23 and the corresponding description for details), the recording software is setup in block 2914 (See FIG. 24 and the corresponding description for details) and the reference activity is recorded in block 2916 (See FIG. 25 and the corresponding description for details). The dose response data is then recorded in block 2918 (See FIG. 30 and the corresponding description for details) and analyzed in block 2920 (See FIG. 32 and the corresponding description for details). If this procedure has not been repeated three times, as determined in decision block 2922, the control culture procedure 2904 is repeated. If, however, the control culture procedure 2904 has been repeated three times, the experimental culture procedure 2924 is performed. For the experimental cultures 2924, the culture is selected in block 2926 (See FIG. 21 and the corresponding description for details), the testing chamber is autoclaved in block 2928 (See FIG. 20 and the corresponding description for details) and the testing chamber is assembled in block 1930 (See FIG. 22 and the corresponding description for details). Thereafter, the recording station is setup in block 2932 (See FIG. 23 and the corresponding description for details), the recording software is setup in block 2934 (See FIG. 24 and the corresponding description for details) and the reference activity is recorded in block 2936 (See FIG. 25 and the corresponding description for details). The dose response data is then recorded in block 2938 (See FIG. 30 and the corresponding description for details) and analyzed in block 2940 (See FIG. 32 and the corresponding description for details). If this procedure has not been repeated three times, as determined in decision block 2942, the experimental culture procedure 2924 (See FIG. 28 and the corresponding description for details) is repeated. If, however, the experimental culture procedure 2924 has been repeated three times, the procedure 2900 ends in block 2946.

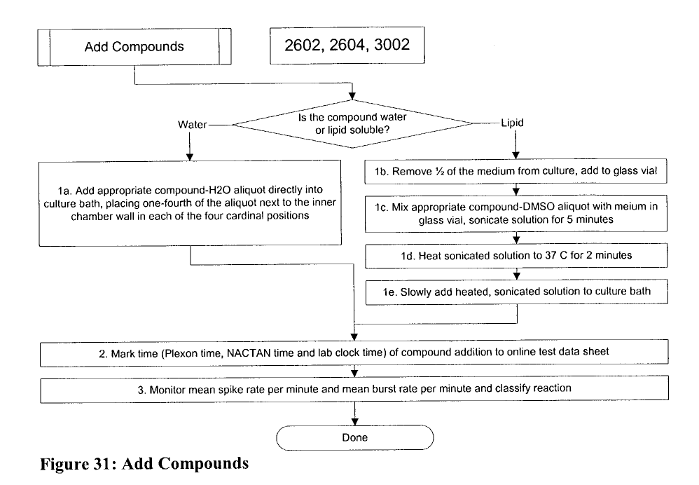

Now referring to FIG. 30, a flow chart describing the procedure to perform the testing needed for the dose response curve development 2918 and 2938 (FIG. 29) is shown. Once the basic neuroactivity difference is shown, a dose response comparison will provide a more quantitative examination of the neuroactivity differences. With both control cultures, usually from a wild-type animal, and an experimental cultures, usually from a genetically modified animal (heterozygous or homozygous), a series of compound concentrations are tested. The procedure for adding the compounds 3002 is shown in FIG. 31). With 5-8 data points, a dose response curve can be developed for both the control culture and experimental culture. The data points are developed from the mean spike and burst rate changes, as a result of specific compound concentrations. The concentration at which there is a 50% response (either excitation or inhibition) is defined as the EC 50. Dose response curves derived from the 5-8 data points can be used to elucidate compound properties such as minimum and maximum effective concentrations, compound efficacy and potency in vitro. The minimum effective concentration is the lowest concentration which will result in a measurable change in network neuroactivity. The maximum effective concentration usually cannot be attained experimentally, but is derived from the maximal plateau on the non-linear curve fitted to the dose response data points (on a semi-log scale). Potency is the position of the fitted curve on the x-axis. A more potent compound will require a lower concentration to elicit a maximal effect, and in turn has a smaller EC50. Efficacy is measured on the y-axis, and is related to the compound's capacity to produce an effect. For example, an inhibitory compound may only inhibit spike rate by 50% at its maximal effective concentration, even if the compound occupies all of the target receptors. In addition, the slope of the dose response curve may reflect mechanism of action and binding properties of the compound.

Referring now to FIG. 31, a flow chart describing the procedure to add compounds to the test cultures in the testing chamber 3002 (FIG. 30) is shown. There is a process to add a compound aliquot to the cell culture chamber. The proper solution should be prepared before adding a compound aliquot. For a water soluble solution, the compound should be directly added to the chamber so as not to cause turbulence in the chamber. For a lipid soluble solution, medium will need to be extracted and mixed with the DMSO-compound solution before it can be slowly added to the chamber. Once added, the times need to be marked on all open programs.

Now referring to FIG. 32, a flow chart describing the procedure to analyze the dose response data and determine the EC 50 2920 and 2940 (FIG. 29) is shown. A Chloroquine concentration-response curve 3200 is also shown.

While this invention has been described in reference to illustrative embodiments, this description is not intended to be construed in a limiting sense. Various modifications and combinations of the illustrative embodiments, as well as other embodiments of the invention, will be apparent to persons skilled in the art upon reference to the description. It is therefore intended that the appended claims encompass any such modifications or embodiments.

What is claimed is:

1. A method for determining the effects of a compound on a neuronal cell comprising the steps of:
growing a first neuronal cell from a control animal and a second neuronal cell from a homozygous genetically modified animal on a first and a second microelectrode, respectively;
exposing the first and second neuronal cells to a compound;
measuring the effects of the compound on the first neuronal cell with the first microelectrode and the second neuronal cell with the second microelectrode; and
comparing the measurements from the first and the second microelectrode to determine the effects of the compound on the neuronal cell.

2. The method of claim 1, wherein the genetically modified cell comprises an embryonic stem cell from a knock-out, knock-in, over-expressing transgenic, under-expressing-transgenic, a conditional knockout, a mutant and the like.

3. The method of claim 1, wherein the genetically modified cell is from an animal knock-out, knock-in, over-expressing transgenic, under-expressing-transgenic, a conditional knockout, a mutant and the like.

4. The method of claim 1, wherein the neuronal cells are selected from the frontal cortex, the auditory cortex, the visual cortex, the hippocampus or the spinal cord.

5. The method of claim 1, wherein the neuronal cells are from a 12-16 day old embryo.

6. The method of claim 1, wherein the neuronal cells are from a heterozygous animal.

7. The method of claim 1, wherein the neuronal cells include one or more types of neuronal cells.

8. The method of claim 1, wherein the neuronal cells form a portion of a neural tissue.

9. The method of claim 1, wherein the neuronal cells are selected from Tables 1-3.

10. A method for determining the effects of a sample on a genetically modified neuronal cell comprising the steps of:
growing a culture of genetically modified neuronal cells on a microelectrode array;
exposing a portion of the genetically modified neuronal cells to a sample; and
measuring the effects of the sample on the genetically modified neuronal cells exposed to the sample to determine the effects of the sample on the genetically modified neuronal cells as compared to results from a like neuronal cell that is not genetically modified.

11. The method of claim 10, wherein the genetically modified cell is from a knock-out, knock-in, over-expressing transgenic, under-expressing-transgenic, a conditional knockout, a mutant and the like embryonic stem cell.

12. The method of claim 10, wherein the genetically modified cell is from a knock-out, knock-in, over-expressing transgenic, under-expressing-transgenic, a conditional knockout, a mutant and the like animal.

13. The method of claim 10, wherein the neuronal cells are from a 12-16 day old embryo.

14. The method of claim 10, wherein the neuronal cells are from a heterozygous animal.

15. The method of claim 10, wherein the neuronal cells include one or more types of neuronal cells.

16. The method of claim 10, wherein the neuronal cells form a portion of a neural tissue.

17. The method of claim 10, wherein the neuronal cells are selected from Tables 1 to 3.

18. The method of claim 10, wherein the neuronal cells are selected from the frontal cortex, the auditory cortex, the visual cortex, the hippocampus or the spinal cord.

19. The method of claim 10, wherein the culture further comprises cells of non-neural origin.

20. An apparatus comprising:
a microelectrode array capable of supporting genetically modified neuronal cells and measuring neuronal activity; and
one or more neuronal cells selected from a genetically modified animal attached to the microelectrode array such that the microelectrode array can record spontaneous neuronal activity of the neuronal cells.

21. The apparatus of claim 20, wherein the genetically modified animal is a knock-out, knock-in, over-expressing transgenic, under-expressing-transgenic, a conditional knockout, a mutant and the like.

22. The apparatus of claim 20, wherein the neuronal cells are from a 12-16 day old embryo.

23. The apparatus of claim 20, wherein the neuronal cells are from a heterozygous animal.

24. The apparatus of claim 20, wherein the neuronal cells include one or more types of neuronal cells.

25. The apparatus of claim 20, wherein the neuronal cells form a portion of a neural tissue.

26. The apparatus of claim 20, wherein the neuronal cells are selected from Tables 1 to 3.

27. The apparatus of claim 20, wherein the neuronal cells are selected from the frontal cortex, the auditory cortex, the visual cortex, the hippocampus or the spinal cord.

28. The apparatus of claim 20, further comprising cells of non-neuronal origin.

29. The apparatus of claim 20, wherein the array is within a recording chamber and is connected to recording equipment.
